(19) United States
(12) Patent Application Publication
(10) Pub. No.: US 2026/0363953 A1
(43) Pub. Date: Nov. 19, 2026

(54) MEDICATION STORAGE AND DISPENSING APPARATUS WITH CONTROLLED SINGLE-CONTAINER ACCESS

(71) Applicant: Smartmed Devices and Services Corporation, Fremont, CA (US)

(72) Inventor: Abdul Raheem Khan, Fremont, CA (US)

(73) Assignee: Smartmed Devices and Services Corporation, Fremont, CA (US)

(21) Appl. No.: 19/678,504

(22) Filed: May 15, 2026

Related U.S. Application Data
(60) Provisional application No. 63/806,095, filed on May 15, 2025.

(51) Int. Cl. A61J 1/14; A61J 7/04; G07F 11/54

(52) U.S. Cl. CPC A61J 1/1437; A61J 7/0463; G07F 11/54

(57) ABSTRACT

A medication storage and dispensing apparatus comprises a housing, a rotatable storage member configured to carry medication containers, and a movable cover movable between closed and open positions. The movable cover selectively exposes a selected medication container at an access region while restricting access to remaining medication containers. A first actuator rotates the rotatable storage member to align the selected medication container with the access region, and a second actuator moves the movable cover between the closed and open positions. In some implementations, the rotatable storage member is reconfigurable between different compartment configurations using removable partitions. A keyed coupler between the first actuator and the rotatable storage member preserves angular registration upon disassembly and reassembly. Force sensors detect load changes corresponding to removal of medication, enabling determination of a weight delta for dispensing verification. Methods of controlled medication dispensing are also disclosed.

### PRIORITY APPLICATION

This application claims the benefit of U.S. Provisional Patent Application No. 63/806,095, filed on May 15, 2025, titled “Smart Medication Adherence System” (Atty. Docket No. SDSC1000USP01) which is hereby incorporated by reference for all purposes.

### TECHNICAL FIELD

The technology disclosed relates to medication storage devices, medication dispensing devices, electromechanical dispensing systems, corresponding control methods, and integrated mechanical and electronic apparatuses for controlled access to stored medications. More particularly, the technology relates to automated medication management, and specifically to systems and methods for storing a plurality of medication containers in a reconfigurable rotatable storage member, selectively exposing a single medication container through a movable cover, and verifying dispensing events through weight-based sensing and controller-driven actuation.

### BACKGROUND

Patients, caregivers, and healthcare providers increasingly rely on medication management devices to support adherence to dosing schedules involving multiple medications taken at different times and in different quantities. Conventional medication organizers, pillboxes, and passive storage trays generally provide little or no control over user access once opened, and commonly permit simultaneous access to multiple medications. As a result, such devices may increase the risk of accidental retrieval of the wrong medication, missed doses, double dosing, or unauthorized access by children or other unintended users. Existing devices also often lack any reliable mechanism for confirming that a medication was actually removed during a scheduled dispensing event.

Existing automated approaches do not adequately address these problems. Many dispensing devices are configured for a fixed number of medication containers and do not readily adapt to changing medication regimens without replacing internal storage structures or replacing the entire device. Other systems may rely only on motor position, door state, or user acknowledgment as a proxy for successful dispensing, without providing a physical verification mechanism tied to actual removal of medication. Still further, certain systems that permit disassembly for refilling, cleaning, or emergency access may lose positional registration upon reassembly and require manual recalibration before reliable dispensing can resume. As a result, an unaddressed gap exists between simple medication organizers and more secure, verifiable, and reconfigurable dispensing systems, and a need remains for an improved technique for controlled medication storage and dispensing that selectively exposes only a chosen medication container, supports multiple installed storage configurations, re-establishes positional reference after reassembly, and verifies dispensing through direct sensing of weight change.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A illustrates a perspective view of a medication storage and dispensing apparatus in an assembled state, according to certain embodiments.

FIG. 1B illustrates an exploded perspective view of the medication storage and dispensing apparatus of FIG. 1A, according to certain embodiments.

FIG. 2 illustrates a perspective view of a top cover assembly of the medication storage and dispensing apparatus, according to certain embodiments.

FIG. 3 illustrates a perspective view of an upper housing of the medication storage and dispensing apparatus, according to certain embodiments.

FIG. 4 illustrates a perspective view of a support structure of the medication storage and dispensing apparatus, according to certain embodiments.

FIG. 5 illustrates a perspective view of a movable cover of the medication storage and dispensing apparatus, according to certain embodiments.

FIG. 6 illustrates a perspective view of a rotatable storage member of the medication storage and dispensing apparatus.

FIG. 7 illustrates a perspective view of a removable partition removably engageable with the rotatable storage member to define a compartment of the medication storage and dispensing apparatus, according to certain embodiments.

FIG. 8 illustrates an exploded perspective view of a female coupler and a male coupler configured to interconnect a first actuator and the rotatable storage member, according to certain embodiments.

FIG. 9 illustrates a perspective view of the first actuator of the medication storage and dispensing apparatus, according to certain embodiments.

FIG. 10 illustrates a sectional view of a drive transmission arrangement of the medication storage and dispensing apparatus in an assembled state, according to certain embodiments.

FIG. 11 illustrates a perspective view of a second actuator of the medication storage and dispensing apparatus, according to certain embodiments.

FIG. 12 illustrates a perspective view of a lighting floor of the medication storage and dispensing apparatus, according to certain embodiments.

FIG. 13 illustrates a perspective view of a lower housing of the medication storage and dispensing apparatus, according to certain embodiments.

FIG. 14 illustrates a perspective view of an actuator mounting bracket supporting the first actuator, according to certain embodiments.

FIG. 15 illustrates a perspective view of a base of the medication storage and dispensing apparatus, the base defining a lower boundary of the apparatus and being configured to receive a plurality of force sensors and a battery, according to certain embodiments.

FIG. 16 illustrates a flow diagram of a method of dispensing medication using the medication storage and dispensing apparatus, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or 5%, and any values therebetween.

### Indicia of Novelty, Inventiveness, Non-Obviousness, and Subject-Matter Eligibility

### Overview

Medication non-adherence is the silent failure of modern healthcare. Approximately half of patients prescribed a chronic medication do not take it as directed. The United States healthcare system absorbs an estimated three hundred billion dollars per year in avoidable cost attributable to that gap. Tens of thousands of children are seen in emergency rooms each year for accidental ingestion of a medication that was never meant to be within reach. The medication is right. The patient is willing. The container in which the medication sits is the bottleneck.

Conventional medication storage answers a different question than care delivery requires. A pillbox organizes medications, but surrenders multiple containers the moment it is opened. An automated dispenser indexes containers, but may not confirm that a container was actually emptied. A locking cabinet restricts access, but may not adapt to a changing regimen. The patient-facing reality is a patchwork of single-purpose devices, none of which solves the medication-delivery problem integrally.

The technology disclosed is a controlled-access medication delivery apparatus engineered to close this gap as a single integrated mechanical system. The apparatus stores a plurality of medication containers in a rotatable storage member. In some embodiments, the apparatus exposes a selected medication container to the user through a powered movable cover. In some embodiments, the apparatus physically restricts access to one or more remaining medication containers during the dispensing event. The apparatus verifies medication removal through a plurality of force sensors angularly distributed beneath the structural load path. The apparatus reconfigures its compartment geometry without disassembly of the storage member from the housing. The apparatus automatically re-establishes its angular reference after disassembly and reassembly without user calibration. In some embodiments, the apparatus generates a tamper-evident audit record of a dispensing event for review by caregivers, clinicians, and compliance systems.

The technology disclosed does not replace the pillbox. The technology disclosed replaces the category. It converts medication storage from a passive organizer into a controlled, verifiable, reconfigurable delivery system suited to hospital inpatient bedsides, long-term-care medication carts, home-health pillbox replacements, controlled-substance dispensing in pharmacy settings, and pediatric households where a single mis-retrieval can be life-threatening.

### How it Works

The apparatus operates in three coordinated phases.

The first phase positions. A first actuator rotates the rotatable storage member about a generally vertical axis to align a selected medication container of a plurality of medication containers carried at angularly distributed positions on the storage member with an access region of the apparatus. The positioning step relies on a keyed female-and-male coupler arrangement that re-establishes a predetermined angular relationship between the rotatable storage member and the first actuator upon disassembly and reassembly of the rotatable storage member from the housing without requiring a user-administered calibration procedure. The first actuator does not seek its position by trial and error; in some embodiments, the coupler's keyed engagement returns the storage member to a known angular registration upon reassembly.

The second phase exposes. A second actuator distinct from the first actuator drives a movable cover about the same generally vertical axis between a closed position obscuring the access region and an open position exposing the access region. The second actuator does not operate the storage member; the storage member's drive train is mechanically isolated from the cover's drive train. The cover carries a plurality of drive features disposed on an outer wall thereof, and a drive member operatively coupled to the second actuator engages those drive features through a complementary meshing engagement. The cover rotates; the storage member does not. The access region is exposed to the user at the angular position the storage member has reached. In some embodiments, one or more non-selected medication containers remain physically blocked during the dispensing event.

The third phase verifies. A plurality of force sensors disposed beneath at least a portion of the housing at angularly distributed positions about the generally vertical axis detects a vertical load transmitted through the structural load path from one or more components of the apparatus supported above the sensors. A baseline reading before the cover opens and a post-dispense reading after the cover closes together yield a measured weight delta. The measured weight delta is compared against the expected weight delta corresponding to the selected medication container. In some embodiments, a controller records the event, the measured delta, and the verification result as a cryptographically signed audit record for caregiver and compliance review.

### The Mechanical Architecture

The technology disclosed rests on six structural elements that work together. Each element contributes to the integrated controlled-access medication delivery described herein.

The first element is two-actuator drive separation with single-compartment exposure. A first actuator rotates the rotatable storage member to align a selected medication container with the access region. A second actuator, distinct from the first actuator, drives the movable cover. The drive paths are mechanically isolated. In some embodiments, the movable cover, in the open position, exposes a selected medication container while restricting user access to one or more remaining medication containers stored in the apparatus. Single-compartment exposure is not a software constraint; it is a physical consequence of the cover geometry, the cover-drive engagement, and the storage-member rotation that, in some embodiments, combine to present a selected compartment to the user. The two-actuator drive separation provides independent control of storage positioning and user access through mechanically distinct drive paths: the first actuator controls rotational alignment of the rotatable storage member, and the second actuator distinct from the first actuator controls movement of the movable cover between the closed position and the open position. In some embodiments, the movable cover, in the open position, exposes a selected medication container while restricting user access to one or more remaining medication containers stored in the apparatus. The two-actuator drive separation enables independent control of the rotatable storage member by the first actuator and independent control of the movable cover by the second actuator, such that storage indexing and user access regulation are mechanically separable functions of the apparatus.

The second element is meshing engagement between the drive features on the outer wall of the movable cover and a complementary drive member. The movable cover carries a plurality of drive features disposed on an outer wall thereof, and the second actuator is operatively coupled to a drive member having a plurality of engagement features complementary to and meshingly engaged with those drive features. The drive engagement is positive, tooth-by-tooth or feature-by-feature contact between two pluralities, not friction-based and not contact-pad-based. Positive engagement enables precise positional control of the cover, predictable holding torque in the closed position, and child-resistant retention without a separate lock. The movable cover is driven through positive meshing engagement between the plurality of drive features disposed on the outer wall of the movable cover and a plurality of engagement features on the drive member of the second actuator. In some embodiments, the meshing engagement provides positional control of the movable cover, retention torque in the closed position, and child-resistant retention of the movable cover without a separate locking mechanism. The meshing engagement is the structural basis for programmatic control of the movable cover by the second actuator and supports both precise positioning of the movable cover between the closed position and the open position and retention of the movable cover in the closed position when the second actuator is in a de-energized state.

The third element is the keyed coupler with calibration-free reassembly. A female coupler is disposed on an underside region of the rotatable storage member; a corresponding male coupler is carried by an output shaft of the first actuator. The male coupler comprises a generally frusto-conical body having a plurality of circumferential engagement features disposed on an outer surface of the frusto-conical body. The female coupler comprises a generally dome-shaped body defining an operative inner cavity configured to receive the male coupler. The cooperation between the circumferential engagement features and the operative inner cavity defines a keyed engagement that re-establishes a predetermined angular relationship between the rotatable storage member and the first actuator upon disassembly and reassembly of the rotatable storage member from the housing without requiring a user-administered calibration procedure. The apparatus tolerates removal of the storage member for cleaning, refilling, emergency access, or service. The keyed engagement between the corresponding male coupler and the female coupler re-establishes a predetermined angular relationship between the rotatable storage member and the first actuator upon disassembly and reassembly of the rotatable storage member from the housing without requiring a user-administered calibration procedure or technician intervention. In some embodiments, the apparatus tolerates removal of the rotatable storage member for cleaning, refilling, emergency access, or service, and returns to operational state upon reassembly without an intervening calibration step. The keyed coupler maintains angular registration between the rotatable storage member and the first actuator across disassembly and reassembly events of the rotatable storage member, and supports operational resumption of the apparatus upon reassembly without an intervening calibration step.

The fourth element is angularly distributed force sensing with battery isolation. A plurality of force sensors is disposed on the upper surface of a base of the apparatus at angularly distributed positions about the generally vertical axis. The plurality of force sensors is configured to detect a vertical load transmitted through the structural load path from one or more components of the apparatus supported above the plurality of force sensors. A battery received within a battery-receiving region defined by upstanding walls of the base is structurally isolated from the load path through the plurality of force sensors. The result is a multi-sensor measurement of the apparatus's structural weight, resolvable to the mass of an individual medication container, and in some embodiments, not affected by battery placement, replacement, or drift. The plurality of force sensors disposed at angularly distributed positions about the generally vertical axis detects a vertical load transmitted through a structural load path of the apparatus from one or more components of the apparatus supported above the plurality of force sensors. In some embodiments, the battery received within the battery-receiving region of the base is structurally isolated from the structural load path through the plurality of force sensors. The angularly distributed sensor arrangement provides multi-point measurement of the structural weight of the apparatus and resolves a measured weight delta corresponding to removal of a selected medication container from the rotatable storage member, the measured weight delta being at least partially independent of battery placement or battery state. The angularly distributed arrangement of the plurality of force sensors enables multi-point measurement of the vertical load transmitted through the structural load path of the apparatus, and the structural isolation of the battery within the battery-receiving region of the base preserves the integrity of the dispensing measurement across battery state changes.

The fifth element is reconfigurable storage geometry without removal of the rotatable storage member. The rotatable storage member comprises a base plate having a plurality of anchor positions disposed circumferentially thereon and a central pillar extending upwardly from the base plate, the central pillar including a plurality of vertical cutouts extending circumferentially about the central pillar. A plurality of removable partitions cooperatively engages the anchor positions on the base plate and the vertical cutouts on the central pillar; each removable partition includes a plurality of engagement legs engageable with a corresponding anchor position and a pillar-engagement edge engageable with a corresponding vertical cutout. In some embodiments, the same physical apparatus supports four-compartment, six-compartment, eight-compartment, or other compartment configurations through the addition or removal of partitions, without disassembling the storage member from the housing and without tools. The apparatus supports a plurality of compartment configurations within the same installed rotatable storage member through addition or removal of one or more removable partitions, without removal of the rotatable storage member from the housing. In some embodiments, the same installed rotatable storage member supports a four-compartment configuration, a six-compartment configuration, or an eight-compartment configuration through reconfiguration of the one or more removable partitions among the plurality of anchor positions on the base plate and the plurality of vertical cutouts on the central pillar. The cooperative engagement among the one or more removable partitions, the plurality of anchor positions on the base plate, and the plurality of vertical cutouts on the central pillar supports reconfiguration of the rotatable storage member among a plurality of compartment configurations through partition placement, without disassembly of the rotatable storage member from the housing.

The sixth element is configuration encoding and verifiable audit-trail generation. A configuration-encoding interface physically encodes the installed compartment configuration of the rotatable storage member; a controller automatically determines the installed configuration upon power-up without manual entry. In some embodiments, a cryptographically signed audit record is generated for a dispensing event, the audit record comprising a medication identifier, a timestamp, the measured weight delta, the expected weight delta, and the verification result. Where the measured delta diverges from the expected delta, a verification failure alert may be generated and transmitted through a network interface to a caregiver, clinician, or compliance system. The configuration-encoding interface physically encodes the installed configuration of the rotatable storage member, such that a controller of the apparatus automatically determines the installed configuration upon power-up based on the physical encoding of the configuration-encoding interface. In some embodiments, the apparatus generates a cryptographically signed audit record for a dispensing event, the audit record comprising a medication identifier, a timestamp, a measured weight delta, an expected weight delta, and a verification result, the audit record being available for remote review through a network interface subsystem of the apparatus. The configuration-encoding interface supports automatic determination of the installed compartment configuration by the controller, and the cryptographic signing of the audit record provides tamper-evident accountability of dispensing events for remote review through the network interface subsystem of the apparatus.

The six elements operate together. The two-actuator drive separation provides the second actuator with independent control of the movable cover. The meshing engagement provides positive transmission between the drive member and the movable cover. The cover, in the open position, can expose a selected compartment that the storage-member positioning has placed under it. The keyed coupler returns the storage member to angular registration after disassembly and reassembly. The force-sensor array verifies that a weight change consistent with removal of the exposed medication has occurred. The audit-trail subsystem ties the verification result to the dispensing event. The configuration encoding supports correspondence between the medication identifier recorded and the physical compartment under the access region. The six elements operate together to produce the integrated controlled-access medication delivery described herein.

### Technical Advantages

The technology disclosed produces a set of concrete, measurable technical advantages, each tied directly to a structural element of the apparatus.

The first advantage is single-compartment access in the open position and physical restriction of access to one or more non-selected compartments. The advantage follows directly from the geometry of the movable cover and the cover's rotation about the same generally vertical axis as the storage member. The advantage is measurable: in some embodiments, a user presented with the apparatus in the open position can reach the medication container exposed at the access region, while access to the remaining containers is restricted.

The second advantage is calibration-free reassembly. The advantage follows directly from the frusto-conical and dome-shaped keyed coupler. In some embodiments, the apparatus returns to operational state after disassembly without a calibration step, a homing screen, a technician dispatch, or a service call. The advantage is measurable: time-to-resume-dispensing after disassembly may be bounded by the time it takes to physically re-seat the storage member, rather than by the time it takes to reset registration.

The third advantage is independent confirmation of medication removal. The advantage follows directly from the angularly distributed force-sensor array beneath the structural load path. A measured weight delta at the sensors confirms that a container was physically removed, independently of motor position, door opening events, encoder reports, or user acknowledgments. The advantage is measurable: a dispensing event may be matched to a sensor-confirmed change in total apparatus mass.

The fourth advantage is reconfigurability across regimens within a single installed device. The advantage follows directly from the central-pillar, base-plate, and removable-partition architecture. In some embodiments, a four-compartment configuration becomes a six-compartment configuration through the addition of partitions without removal of the storage member, without tools, and without replacement of the device. The advantage is measurable: the unit cost of a regimen change may be bounded by the cost of one or more partitions, rather than by the cost of a new device.

The fifth advantage is structurally isolated weight verification. The advantage follows directly from the battery-isolated load path on the base. In some embodiments, battery insertion, removal, or replacement does not contribute to the vertical load detected at the plurality of force sensors, and changes in battery state do not propagate into the dispensing measurement. The advantage is measurable: the weight delta corresponding to a dispensing event may remain substantially invariant under battery state changes.

The sixth advantage is automatic determination of installed compartment geometry. The advantage follows directly from the configuration-encoding interface and the controller's startup routine. In some embodiments, a change in the installed compartment configuration does not require a setup screen, a user manual lookup, or a software configuration step; the controller reads the configuration from the physical encoding on power-up. The advantage is measurable: configuration-mismatch errors may be reduced independent of operator skill.

The seventh advantage is a tamper-evident audit trail tied to physical dispensing events. The advantage follows directly from the cryptographic signing of the audit record and the network-interface transmission of verification failures. In some embodiments, a caregiver, clinician, or compliance system has access to a structured, signed, time-stamped record of dispensing events and verification failures. The advantage is measurable: post-event reconciliation is performed by reviewing the audit log.

Each of the advantages above follows from a structural element described herein, and each is measurable in operation. Together, these advantages distinguish the disclosed apparatus from passive medication-storage devices.

### Why it Matters

Medication non-adherence and unintended medication access are not problems of patient willingness or pharmaceutical efficacy. They are problems of the container in which the medication sits. The pillbox, the bottle organizer, the locking cabinet, and the single-actuator dispenser each address a fraction of the medication-delivery problem and leave the remainder unsolved.

The technology disclosed is not an incremental improvement to existing pillboxes or organizers. The technology disclosed is the structural step that converts medication storage from a passive container into a controlled, verifiable, reconfigurable delivery system. The apparatus stores. In some embodiments, the apparatus exposes one medication at a time. The apparatus restricts access to the rest. The apparatus verifies removal through direct sensing. The apparatus tolerates disassembly without losing its registration. The apparatus reconfigures itself for changing regimens without being replaced. The apparatus records what it did, when it did it, and whether the medication was removed.

The medication that gets dispensed may be the medication that gets verified. The dispensing event that gets verified may be the dispensing event that gets logged. The audit record created today may be the audit record reviewed tomorrow.

The apparatus is suited to settings in which medication delivery matters and unverified delivery is undesirable: hospital inpatient bedsides where the wrong pill has clinical consequences, long-term-care facilities where a caregiver may oversee dozens of regimens, home-health environments where a remote clinician cannot observe each dose, pediatric households where a single mis-retrieval can be life-threatening, and controlled-substance pharmacy settings where the chain of custody matters as much as the dispensing itself.

The technology disclosed is not a productivity enhancement. It is the architectural step that closes the gap between the medication that is prescribed and the medication that is actually taken.

### Statement of Subject-Matter Eligibility

The technology disclosed is directed to a particular technical solution to a particular technical problem and is rooted in specific structural elements, actuator arrangements, sensor architectures, and control operations. The technology disclosed is not directed to an abstract idea, a mental process, or an unspecified application of generic computing. The technology disclosed operates through physical movement and sensing of structural components of a medication storage and dispensing apparatus.

The operative steps of the technology disclosed are executed through coordinated operation of a first actuator rotating a rotatable storage member, a second actuator moving a movable cover, a keyed coupler preserving angular registration through cooperation between a frusto-conical male body and a dome-shaped female cavity, an angular reference feature cooperating with a fixed reference structure to define a home angular reference, a configuration-encoding interface physically encoding an installed compartment configuration, and a plurality of force sensors supported relative to a base and configured to detect a vertical load transmitted from one or more components of the apparatus supported above the base. A human user does not manually reproduce the claimed combination of single-compartment exposure, motor-driven blocking of remaining compartments, physical encoding of installed compartment geometry, automatic post-reassembly homing through keyed re-engagement, and weight-based verification of medication removal performed through the force-sensor arrangement of the apparatus.

The technology disclosed produces tangible physical effects in the apparatus and in the stored medication environment. The technology disclosed physically repositions the rotatable storage member, physically moves the movable cover to alter user access to medication containers, physically constrains access to non-selected medication containers, and physically detects changes in weight corresponding to medication removal through the plurality of force sensors supported relative to the base. The technology disclosed therefore presents a particular technical solution to a particular technical problem, requires operation of defined mechanical and electromechanical components, and improves the functioning of a medication storage and dispensing apparatus itself.

### Overall Apparatus Overview

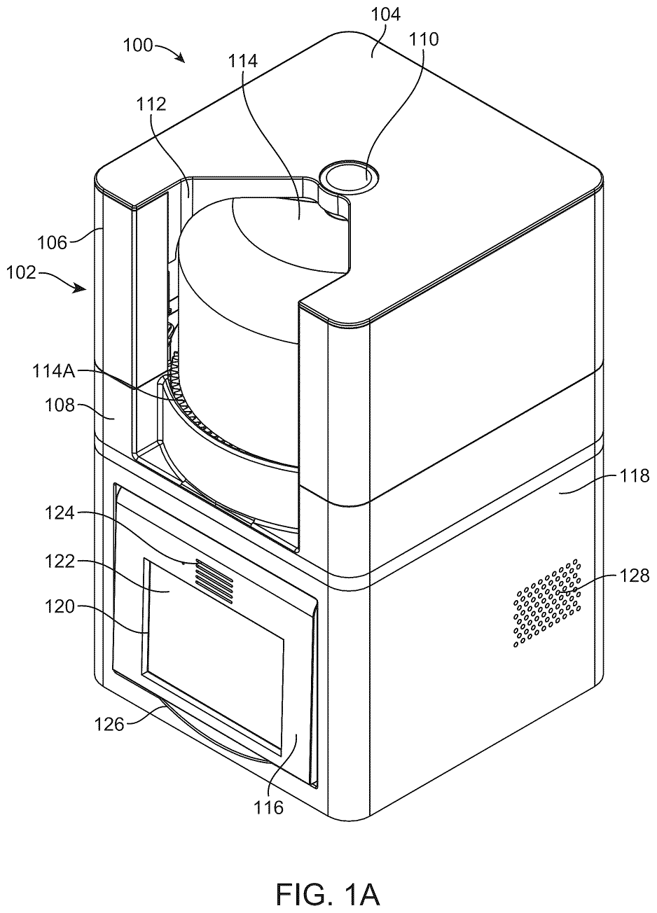

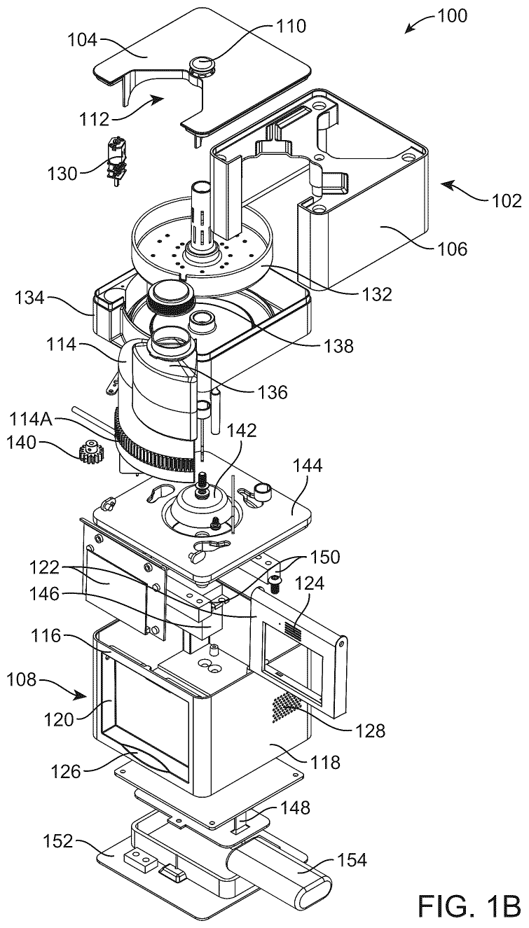

FIG. 1A illustrates a perspective view of a medication storage and dispensing apparatus 100 in an assembled state. In the illustrated embodiment, the apparatus 100 is shown as a compact, upright unit having a generally rectilinear overall form with rounded vertical corner regions and a vertically stacked arrangement of upper and lower portions. The apparatus 100 includes a housing 102. In the illustrated embodiment, the housing 102 includes a top cover assembly 104, an upper housing 106, and a lower housing 108. The housing 102 defines the visible exterior of the apparatus 100 and encloses internal components of the apparatus 100 while leaving selected user-facing and user-accessible regions exposed.

The top cover assembly 104 is disposed at an upper end of the apparatus 100 and defines an upper boundary of the housing 102. In the illustrated embodiment, the top cover assembly 104 has a generally planar upper surface extending across substantially the full width and depth of the apparatus 100, with rounded peripheral corner regions corresponding generally to the contour of the apparatus 100 as a whole. The top cover assembly 104 is shown as a single visible upper member mounted above the upper housing 106. Although the top cover assembly 104 is shown with a substantially planar upper surface, the top cover assembly 104 may, in other embodiments, include one or more raised portions, recessed portions, contour features, textured portions, reinforcement features, sealing features, or fastening features, and the particular geometry shown in FIG. 1A is illustrative and not limiting.

A screw cap 110 is disposed on the top cover assembly 104. In the illustrated embodiment, the screw cap 110 is positioned at a front region of the top cover assembly 104 and covers a fastening interface between the top cover assembly 104 and the upper housing 106. The screw cap 110 conceals a fastener, such as a screw, extending through the top cover assembly 104 into the upper housing 106, although the fastener itself is not visible in FIG. 1A. The screw cap 110 appears as a small generally circular feature visible from above. In other embodiments, the screw cap 110 may comprise a snap-in cap, a press-fit cap, a threaded cap, a removable cap, a fixed cover feature, or any other structure suitable for concealing or partially concealing a fastening interface. In still other embodiments, the screw cap 110 may be omitted altogether. Accordingly, the particular form of the screw cap 110 shown in FIG. 1A is illustrative and not limiting.

The top cover assembly 104 further defines a cutout 112. In the illustrated embodiment, the cutout 112 is positioned at a front region of the top cover assembly 104 and extends inwardly from a front edge region of the top cover assembly 104. The cutout 112 interrupts an otherwise continuous front portion of the top cover assembly 104 and provides visual and physical access to an upper dispensing region of the apparatus 100. As shown in FIG. 1A, the cutout 112 has a generally recessed profile and is shaped to expose a portion of a movable cover 114 positioned below the top cover assembly 104. Although the cutout 112 is shown with a particular curved and recessed geometry, the cutout 112 may, in other embodiments, have any other suitable shape, including an arcuate shape, a circular shape, an oval shape, a slot-like shape, a polygonal shape, or a compound profile. The particular shape and extent of the cutout 112 shown in FIG. 1A is therefore illustrative and not limiting.

The movable cover 114 is visible at an upper front region of the apparatus 100. In the illustrated embodiment, the movable cover 114 is positioned within the housing 102 below the top cover assembly 104 and is exposed through the cutout 112. The visible portion of the movable cover 114 has a generally curved outer profile and a generally cylindrical or partially cylindrical appearance when viewed in perspective. The movable cover 114 is arranged about a generally vertical axis and occupies a central portion of an upper region of the apparatus 100. Only a portion of the movable cover 114 is visible in FIG. 1A because the top cover assembly 104 and the upper housing 106 partially overlie and surround the movable cover 114.

In the illustrated embodiment, the movable cover 114 includes a plurality of drive features 114A on an outer wall of the movable cover 114. The plurality of drive features 114A is visible as a toothed region extending along a lower circumferential portion of the movable cover 114. The plurality of drive features 114A is engageable with an actuator, not visible in FIG. 1A, to move the movable cover 114 between positions corresponding to closed and open access states of the apparatus 100. In some embodiments, the plurality of drive features 114A may comprise gear teeth. In other embodiments, the plurality of drive features 114A may comprise ridges, engagement projections, frictional drive features, belt-engagement features, magnetic drive features, or any other drive-transmission features suitable for moving the movable cover 114. Rotation or other driven movement of the movable cover 114 selectively permits access to a selected medication container held within the apparatus 100. The visible representation of the movable cover 114 in FIG. 1A is intended to show its general position and general external profile and is not intended to limit the internal structure, opening arrangement, or drive arrangement of the movable cover 114.

The upper housing 106 is disposed below the top cover assembly 104 and above the lower housing 108. In the illustrated embodiment, the upper housing 106 defines an upper structural region of the apparatus 100 surrounding the movable cover 114 and enclosing the upper dispensing region of the apparatus 100. The upper housing 106 includes visible side wall portions and front wall portions extending downwardly from the top cover assembly 104. The upper housing 106 cooperates with the top cover assembly 104 to define a partially enclosed upper region of the apparatus 100 within which the movable cover 114 is located. The upper housing 106 is shown with generally vertical wall portions and rounded corner transitions, although other wall geometries may be used in other embodiments.

The lower housing 108 is disposed beneath the upper housing 106 and defines the lower portion of the apparatus 100. In the illustrated embodiment, the lower housing 108 has a generally box-like visible form with rounded vertical corner regions and generally planar front and side wall regions. A generally horizontal boundary between the upper housing 106 and the lower housing 108 is visible in FIG. 1A, thereby visually separating the upper and lower portions of the apparatus 100. The lower housing 108 may house electronic, sensing, power, and control components of the apparatus 100, although such internal components are not visible in FIG. 1A.

The lower housing 108 includes a front face 116. The front face 116 defines an opening 120 receiving a user-interface display 122. In the illustrated embodiment, the opening 120 is positioned in a lower front region of the apparatus 100 and has a generally rectangular profile. The user-interface display 122 is shown received within the opening 120 and faces generally forwardly for viewing by a user. In other embodiments, the user-interface display 122 may comprise any suitable display arrangement, including a touchscreen display, a liquid crystal display, an organic light-emitting diode display, a segmented display, or another display structure.

A plurality of apertures 124 is positioned above the opening 120 and above the user-interface display 122 on the front face 116. In the illustrated embodiment, the plurality of apertures 124 is arranged in a narrow generally horizontal grouping. The plurality of apertures 124 may provide acoustic transmission, ventilation, or both. A recess 126 is positioned below the opening 120 receiving the user-interface display 122. In the illustrated embodiment, the recess 126 has a generally curved profile and is located at a lower front region of the lower housing 108. The recess 126 may provide a finger-engagement feature, a contour feature, or another localized surface feature.

The lower housing 108 further includes a side face 118. The side face 118 is visible on the right side of the apparatus 100 in the perspective view of FIG. 1A. A plurality of perforations 128 is disposed on the side face 118. In the illustrated embodiment, the plurality of perforations 128 is arranged in a grouped array of small openings. The plurality of perforations 128 may provide ventilation, acoustic transmission, or both. In other embodiments, the plurality of perforations 128 may be replaced by slots, meshes, louvers, or other opening structures.

As shown in FIG. 1A, the apparatus 100 has a vertically organized arrangement in which the movable cover 114 is positioned in the upper portion of the apparatus 100 while the opening 120 receiving the user-interface display 122 is positioned in the lower portion of the apparatus 100. The top cover assembly 104, upper housing 106, and lower housing 108 cooperate to define a compact freestanding device having an upper medication-access region and a lower user-interface region. The apparatus 100 is shown in a closed and assembled state in which the housing 102 encloses internal components of the apparatus 100 and exposes selected external features for user interaction and controlled medication access.

FIG. 1B illustrates an exploded perspective view of the medication storage and dispensing apparatus 100 of FIG. 1A. In the illustrated embodiment, the components of the apparatus 100 are shown in vertically separated relation along a generally vertical assembly axis, thereby illustrating the relative order in which the components are arranged in the assembled state of FIG. 1A. Components previously described with reference to FIG. 1A, including the top cover assembly 104, the screw cap 110, the cutout 112, the movable cover 114, the plurality of drive features 114A, the upper housing 106, the lower housing 108, the front face 116, the side face 118, the opening 120 receiving the user-interface display 122, the plurality of apertures 124, the recess 126, and the plurality of perforations 128, are shown again in FIG. 1B in exploded relation. FIG. 1B further shows internal components of the apparatus 100 not separately visible, or not fully visible, in FIG. 1A, including a second actuator 130, a rotatable storage member 132, a support structure 134, a medication container 136, a child-resistant cap 138, a drive member 140, a female coupler 142, a lighting floor 144, a first actuator 146, an actuator mounting bracket 148, a plurality of force sensors 150, a base 152, and a battery 154.

The top cover assembly 104 is shown at an upper end of the exploded view of FIG. 1B above the remaining components of the apparatus 100, with the screw cap 110 and the cutout 112 visible as previously described with reference to FIG. 1A. In the assembled state of FIG. 1A, the top cover assembly 104 is mounted above the upper housing 106 such that the cutout 112 of the top cover assembly 104 exposes a portion of the movable cover 114 received within the upper housing 106. In other embodiments, the top cover assembly 104 may be removably mounted on, hingedly mounted to, or otherwise coupled to the upper housing 106 using one or more fasteners, snap features, magnetic features, or other attachment arrangements suitable for retaining the top cover assembly 104 on the upper housing 106.

The second actuator 130 is shown below the top cover assembly 104 and laterally offset from the upper housing 106 in the exploded view of FIG. 1B. In the illustrated embodiment, the second actuator 130 has a generally compact form and is positioned in the upper region of the apparatus 100. The second actuator 130 is mechanically coupled to the movable cover 114 by way of the drive member 140 described below and is configured to move the movable cover 114 between a closed position obscuring an access region of the apparatus 100 and an open position exposing the access region. In a de-energized state, the second actuator 130 retains the movable cover 114 in the closed position with a retention torque sufficient to resist user attempts to manually displace the movable cover 114, thereby providing a child-resistant retention mechanism that operates independently of an angular position of the rotatable storage member 132. In some embodiments, the second actuator 130 comprises a direct-current motor. In other embodiments, the second actuator 130 may comprise a stepper motor, a servo motor, a geared motor, a brushless motor, a linear actuator coupled to a rotary mechanism, a solenoid-driven actuator, or another electromechanical drive element suitable for moving the movable cover 114. The particular external form of the second actuator 130 shown in FIG. 1B is illustrative and not limiting.

The upper housing 106 is shown laterally offset from the second actuator 130 and below the top cover assembly 104 in the exploded view of FIG. 1B. As described with reference to FIG. 1A, the upper housing 106 defines the upper structural region of the apparatus 100. In FIG. 1B, the upper housing 106 is shown separately from the internal components received therein, thereby exposing an upper interior volume of the upper housing 106. The upper interior volume is sized to receive the support structure 134, the rotatable storage member 132, and the movable cover 114 therewithin in the assembled state of FIG. 1A. The upper housing 106 includes one or more interior locating features visible on an inner wall thereof, the interior locating features being configured to position the support structure 134 and other internal components in a predetermined arrangement within the upper housing 106.

The support structure 134 is shown below the second actuator 130 and laterally adjacent the upper housing 106 in the exploded view of FIG. 1B. In the illustrated embodiment, the support structure 134 has a generally cup-shaped or annular configuration and is sized to receive the rotatable storage member 132 from above. In the assembled state of FIG. 1A, the support structure 134 is fixedly mounted within the upper housing 106 such that the support structure 134 does not rotate, while the rotatable storage member 132 is received by and rotatable relative to the support structure 134 about the generally vertical axis. In other embodiments, the support structure 134 may be formed as a unitary body or as a multi-piece assembly, and may comprise polymeric, metallic, composite, or combination materials. The particular geometry of the support structure 134 shown in FIG. 1B is illustrative and not limiting.

The rotatable storage member 132 is shown below the support structure 134 in the exploded view of FIG. 1B. In the illustrated embodiment, the rotatable storage member 132 includes a generally circular base plate having a plurality of anchor positions disposed circumferentially thereon and a central pillar extending upwardly from the base plate, the central pillar including a plurality of vertical cutouts extending circumferentially about the central pillar. The plurality of anchor positions and the plurality of vertical cutouts are arranged to cooperatively receive one or more removable partitions (not visible in FIG. 1B), the one or more removable partitions being removably engageable with the rotatable storage member 132 to define, in cooperation with the base plate and the central pillar of the rotatable storage member 132, one or more compartments each configured to receive a medication container 136. In the assembled state of FIG. 1A, the rotatable storage member 132 is received within the support structure 134 and is rotatable relative to the support structure 134 about the generally vertical axis. The rotatable storage member 132 is configured to be rotated by the first actuator 146 through the female coupler 142 described below. Rotation of the rotatable storage member 132 aligns a selected medication container 136, from among a plurality of medication containers carried by the rotatable storage member 132, with the access region of the apparatus 100. The rotatable storage member 132 is reconfigurable between a first configuration having a first number of compartments and a second configuration having a second number of compartments different from the first number, the reconfiguration being effected by addition or removal of one or more of the removable partitions without removal of the rotatable storage member 132 from the housing 102. In other embodiments, the rotatable storage member 132 may have a circular plan shape, an annular plan shape, a segmented plan shape, or another rotatable arrangement suitable for supporting a plurality of medication containers at angularly distributed positions.

The medication container 136 and the child-resistant cap 138 are shown adjacent the rotatable storage member 132 in the exploded view of FIG. 1B. The medication container 136 is also referred to herein as a vial or as a medicine container. In the illustrated embodiment, the medication container 136 has a generally cylindrical body and an upper neck region, and the child-resistant cap 138 is removably mountable on the upper neck region of the medication container 136. In the assembled state of FIG. 1A, the medication container 136 is receivable within a compartment defined by the rotatable storage member 132 and a corresponding removable partition, and the child-resistant cap 138 is mounted on the medication container 136 to provide a child-resistant closure requiring a deliberate combined manipulation by a user to disengage the child-resistant cap 138 from the medication container 136. Although a single medication container 136 and a single child-resistant cap 138 are shown in FIG. 1B for clarity, the apparatus 100 is configured to receive a plurality of medication containers 136 in corresponding compartments of the rotatable storage member 132. In other embodiments, the child-resistant cap 138 may comprise a press-and-turn closure, a squeeze-and-turn closure, a lift-and-turn closure, or another closure arrangement configured to require a deliberate combined manipulation by a user.

In some embodiments, the apparatus 100 is configured such that medication is manually removed from the medication container 136 after the medication container 136 is exposed through the access region. In other embodiments, the apparatus 100 further includes an automated dispensing mechanism configured to cooperate with the medication container 136 to dispense medication therefrom under control of the controller. The automated dispensing mechanism may operate while the medication container 136 remains supported by the rotatable storage member 132 or after partial presentation of the medication container 136 at the access region. The particular structure of the automated dispensing mechanism may vary across embodiments and is not limited to any specific dispensing arrangement unless expressly recited. The automated dispensing mechanism is not separately shown in FIG. 1B.

The movable cover 114 is shown below the rotatable storage member 132 in the exploded view of FIG. 1B. As described with reference to FIG. 1A, the movable cover 114 is visible externally through the cutout 112 of the top cover assembly 104 in the assembled state. In FIG. 1B, the movable cover 114 is shown more fully than in FIG. 1A, illustrating a generally cylindrical or partially cylindrical body extending around the access region of the apparatus 100 and the plurality of drive features 114A disposed on the outer wall of the movable cover 114. The plurality of drive features 114A is shown as a toothed circumferential region along a lower portion of the outer wall. The movable cover 114 is rotatable about the generally vertical axis to move between the closed position obscuring the access region and the open position exposing the access region such that a selected medication container 136 within the rotatable storage member 132 is accessible to a user. In some embodiments, the plurality of drive features 114A comprises a plurality of gear teeth. In other embodiments, the plurality of drive features 114A may comprise belt-engaging ridges, friction-engaging surface features, magnetic drive features, or any combination thereof.

The drive member 140 is shown adjacent the movable cover 114 in the exploded view of FIG. 1B. In the illustrated embodiment, the drive member 140 has a generally cylindrical form with peripheral drive features configured to engage the plurality of drive features 114A on the outer wall of the movable cover 114. The drive member 140 is mechanically coupled to an output of the second actuator 130 and transmits rotational motion from the second actuator 130 to the movable cover 114. In the assembled state of FIG. 1A, the drive member 140 is positioned adjacent the outer wall of the movable cover 114 such that engagement of the drive member 140 with the plurality of drive features 114A moves the movable cover 114 between the closed position and the open position upon actuation of the second actuator 130. In other embodiments, the drive member 140 may comprise a pinion gear, a friction wheel, a pulley, a roller, a magnetic coupler, or another driven transmission element, and the engagement between the drive member 140 and the plurality of drive features 114A may be meshed, frictional, magnetic, or otherwise mechanically coupled.

The female coupler 142 is visible on an underside region of the rotatable storage member 132 in the exploded view of FIG. 1B. In the illustrated embodiment, the female coupler 142 is generally coaxial with the generally vertical axis and includes an alignment feature configured to constrain angular engagement with a corresponding male coupler (not visible in FIG. 1B) carried by an output shaft of the first actuator 146. In the assembled state of FIG. 1A, the female coupler 142 receives the corresponding male coupler from below such that rotation imparted by the first actuator 146 is transmitted to the rotatable storage member 132 through a keyed engagement between the female coupler 142 and the corresponding male coupler. The keyed engagement re-establishes a predetermined angular relationship between the rotatable storage member 132 and the first actuator 146 upon each disassembly and reassembly of the rotatable storage member 132 from the housing 102, without requiring a user-administered calibration procedure. In other embodiments, the female coupler 142 and the corresponding male coupler may comprise mating polygonal profiles, mating keyed profiles, mating spline profiles, mating dog-clutch profiles, or any other angularly constrained mating arrangement suitable for transmitting rotational torque while preserving an angular reference between the rotatable storage member 132 and the first actuator 146.

The lighting floor 144 is shown below the movable cover 114 and below the support structure 134 in the exploded view of FIG. 1B. In the illustrated embodiment, the lighting floor 144 has a generally plate-like form extending laterally beneath the upper dispensing region of the apparatus 100. The lighting floor 144 defines a central aperture aligned generally with the generally vertical axis. In the assembled state of FIG. 1A, the lighting floor 144 is positioned between the support structure 134 above and the lower housing 108 below, and the central aperture of the lighting floor 144 is configured to receive the output shaft of the first actuator 146 therethrough for engagement with the female coupler 142 on the underside region of the rotatable storage member 132. In some embodiments, the lighting floor 144 carries one or more light sources configured to selectively illuminate compartments of the rotatable storage member 132 from below. In other embodiments, the lighting floor 144 may include one or more light-transmissive portions, one or more diffusing portions, one or more reflective portions, or any combination thereof, and may be formed from polymeric, metallic, composite, or combination materials. The particular form of the lighting floor 144 shown in FIG. 1B is illustrative and not limiting.

The first actuator 146 is shown below the lighting floor 144 in the exploded view of FIG. 1B. In the illustrated embodiment, the first actuator 146 is aligned generally with the generally vertical axis and is configured to impart rotation to the rotatable storage member 132 through the female coupler 142 and the corresponding male coupler. In the assembled state of FIG. 1A, the first actuator 146 is supported within the lower housing 108 such that the output shaft of the first actuator 146 extends upwardly through the central aperture of the lighting floor 144 and engages the female coupler 142 on the underside region of the rotatable storage member 132. The first actuator 146 is distinct from the second actuator 130, such that rotation of the rotatable storage member 132 and movement of the movable cover 114 are independently controllable. In other embodiments, the first actuator 146 may comprise a stepper motor, a brushless direct-current motor, a brushed direct-current motor, a servo motor, an alternating-current motor, or another electromechanical drive element configured to impart controlled angular displacement to the rotatable storage member 132.

The lower housing 108 is shown below the first actuator 146 in the exploded view of FIG. 1B. As described with reference to FIG. 1A, the lower housing 108 includes the front face 116 with the opening 120 receiving the user-interface display 122, the plurality of apertures 124, the recess 126, and the side face 118 with the plurality of perforations 128. In FIG. 1B, the lower housing 108 is shown in exploded relation to components positioned above and below it, thereby exposing an interior volume of the lower housing 108 sized to receive the first actuator 146 and electrical components associated with operation of the apparatus 100. In other embodiments, the lower housing 108 may comprise a unitary molded structure, a multi-piece assembly, a metallic structure, a polymeric structure, or any combination thereof.

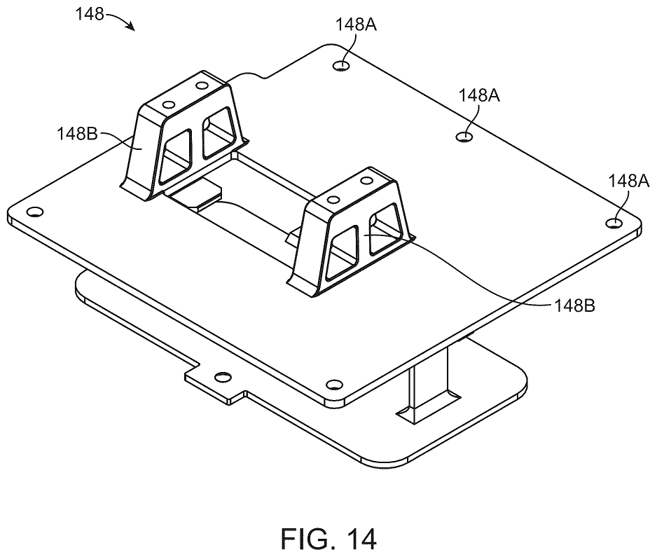

The actuator mounting bracket 148 is shown below the lower housing 108 in the exploded view of FIG. 1B. In the illustrated embodiment, the actuator mounting bracket 148 has a multi-plate configuration. In the assembled state of FIG. 1A, the actuator mounting bracket 148 is disposed within the interior volume of the lower housing 108 to receive and retain the first actuator 146 in a predetermined orientation as further described with reference to FIG. 14.

The plurality of force sensors 150 is shown in the exploded view of FIG. 1B. The plurality of force sensors 150 is also referred to herein as a plurality of load cells. In the illustrated embodiment, the plurality of force sensors 150 is arranged at angularly distributed positions about the generally vertical axis. Each force sensor of the plurality of force sensors 150 is configured to detect a portion of a vertical load transmitted to the base 152, the plurality of force sensors 150 cooperating to detect a total vertical load. In some embodiments, the plurality of force sensors 150 comprises four force sensors disposed at four cardinal positions about the generally vertical axis. In other embodiments, the plurality of force sensors 150 may comprise three force sensors, four force sensors, six force sensors, or any other number of force sensors sufficient to detect the total vertical load, and may comprise strain-gauge load cells, piezoelectric force sensors, capacitive force sensors, optical force sensors, magnetostrictive force sensors, or any combination thereof.

The battery 154 is shown in the exploded view of FIG. 1B. In the illustrated embodiment, the battery 154 has a generally cylindrical body and is configured to provide electrical power to the first actuator 146, the second actuator 130, and other electrical components of the apparatus 100. In other embodiments, the battery 154 may comprise a lithium-ion battery, a lithium-polymer battery, a nickel-metal hydride battery, an alkaline battery, or another portable electrical power source, and may comprise a single battery cell or a plurality of battery cells connected in series, in parallel, or in a series-parallel combination.

The base 152 is shown at a lower end of the exploded view of FIG. 1B. In the illustrated embodiment, the base 152 has a generally planar form and forms a lower boundary of the apparatus 100. The base 152 has an upper surface configured to receive the plurality of force sensors 150 thereon and a lower surface configured to seat upon a horizontal support surface in the assembled state of FIG. 1A. In other embodiments, the base 152 may comprise a unitary molded body, a fabricated assembly, a plate-and-frame structure, a footed structure including a plurality of feet disposed on the lower surface, or another support structure configured to seat upon a horizontal support surface and to receive the plurality of force sensors 150.

In the illustrated embodiment, the components of the apparatus 100 shown in the exploded view of FIG. 1B are vertically arranged along the assembly axis such that, in the assembled state of FIG. 1A, the base 152 supports the plurality of force sensors 150, the plurality of force sensors 150 supports the lower housing 108, the actuator mounting bracket 148 is disposed within the lower housing 108 and receives and retains the first actuator 146, the lighting floor 144 is positioned above the lower housing 108 and defines the central aperture through which the output shaft of the first actuator 146 extends to engage the female coupler 142 on the underside region of the rotatable storage member 132, the support structure 134 is fixedly mounted within the upper housing 106 above the lighting floor 144, the rotatable storage member 132 is received within the support structure 134, the movable cover 114 is rotatable about the rotatable storage member 132 and is engaged by the drive member 140 driven by the second actuator 130, and the top cover assembly 104 is removably mounted at an upper end of the upper housing 106 above the rotatable storage member 132 and the movable cover 114.

It will be appreciated that the foregoing arrangement of components shown in the exploded view of FIG. 1B is illustrative of one configuration of the apparatus 100 and is not intended to be limiting. In other embodiments, the apparatus 100 may include additional components not shown in FIG. 1B, may omit one or more of the components shown in FIG. 1B, or may arrange one or more of the components in a different vertical or angular relationship without departing from the scope of the disclosed technology. Components shown in FIG. 1B as discrete elements may be combined into a unitary structure in other embodiments, and components shown in FIG. 1B as unitary structures may be subdivided into discrete elements in other embodiments. References to “above” and “below” in connection with FIG. 1B refer to the vertically exploded relationship illustrated and are not intended to limit the relative position of the components in the assembled state of FIG. 1A or in any other embodiment.

Accordingly, FIG. 1B illustrates the medication storage and dispensing apparatus 100 in exploded relation and shows, by way of example, the top cover assembly 104, the screw cap 110, the cutout 112, the second actuator 130, the upper housing 106, the support structure 134, the rotatable storage member 132, the medication container 136, the child-resistant cap 138, the movable cover 114, the plurality of drive features 114A, the drive member 140, the female coupler 142, the lighting floor 144, the first actuator 146, the lower housing 108 with the front face 116, the side face 118, the opening 120 receiving the user-interface display 122, the plurality of apertures 124, the recess 126, and the plurality of perforations 128 as previously described with reference to FIG. 1A, the actuator mounting bracket 148, the plurality of force sensors 150, the base 152, and the battery 154. The embodiment shown in FIG. 1B is exemplary only, and the relative shapes, proportions, positions, and assembly relationships of the illustrated components may be varied in other embodiments without departing from the scope of the disclosed technology.

### Top Cover

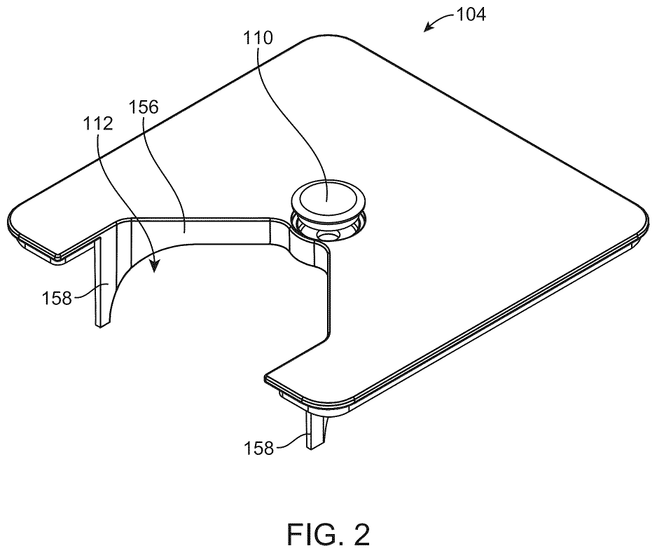

FIG. 2 illustrates a perspective view of the top cover assembly 104 of the medication storage and dispensing apparatus 100. As described with reference to FIG. 1A and FIG. 1B, the top cover assembly 104 is disposed at the upper end of the apparatus 100 and is mounted above the upper housing 106 in the assembled state of FIG. 1A. In FIG. 2, the top cover assembly 104 is shown as a discrete component such that the upper surface of the top cover assembly 104 and an underside of the top cover assembly 104 are both visible.

In the illustrated embodiment, the top cover assembly 104 has a generally planar body with a generally rectangular peripheral profile interrupted by the cutout 112, and has a generally thin thickness, such that the top cover assembly 104 forms a low-profile upper member of the housing 102. The screw cap 110 is visible at the front region of the top cover assembly 104 as previously described with reference to FIG. 1A. The cutout 112 is visible at the front region of the top cover assembly 104 as previously described with reference to FIG. 1A, extending inwardly from the front edge region of the top cover assembly 104. In FIG. 2, the cutout 112 is shown more fully than in FIG. 1A, illustrating a generally curved inner boundary at one side of the cutout 112 and a generally linear boundary at another side of the cutout 112.

A depending wall 156 extends downwardly from the underside of the top cover assembly 104 along the inner boundary of the cutout 112. In the illustrated embodiment, the depending wall 156 follows generally the curved inner boundary of the cutout 112 and projects a predetermined distance downwardly relative to the underside of the top cover assembly 104. The depending wall 156 cooperates with the cutout 112 to define a recessed enclosure extending below the upper surface of the top cover assembly 104. In the assembled state of FIG. 1A, the depending wall 156 extends downwardly adjacent the movable cover 114 and partially overlies a region adjacent the movable cover 114. In other embodiments, the depending wall 156 may comprise a generally curved wall portion, a generally planar wall portion, a multi-faceted wall portion, or any combination thereof, and may be formed as an integrally molded extension of the top cover assembly 104, as a separately formed wall affixed to the underside of the top cover assembly 104, or as any other downwardly extending structure suitable for partially enclosing a region below the cutout 112.

A plurality of depending projections 158 extends downwardly from the underside of the top cover assembly 104 at spaced positions relative to the depending wall 156. In the illustrated embodiment, the plurality of depending projections 158 includes a first depending projection and a second depending projection disposed on opposite sides of the cutout 112 and projecting downwardly to engage the upper housing 106 in the assembled state of FIG. 1A. The plurality of depending projections 158 is configured to position the top cover assembly 104 in a predetermined orientation relative to the upper housing 106 and to cooperate with the upper housing 106 and with the fastening interface concealed by the screw cap 110 to retain the top cover assembly 104 on the upper housing 106. In other embodiments, the plurality of depending projections 158 may comprise two depending projections, three depending projections, four depending projections, or any other number of depending projections sufficient to position and retain the top cover assembly 104 on the upper housing 106, and may comprise depending tabs, depending posts, depending legs, snap-fit features, alignment pins, locating pins, friction-fit features, or any combination thereof, and may be integrally formed with or separately attached to the top cover assembly 104.

In the illustrated embodiment, the top cover assembly 104 of FIG. 2 is shown as an integrally formed component. In other embodiments, the top cover assembly 104 may comprise multiple joined sub-components and may be molded, machined, fabricated, cast, thermoformed, additively manufactured, or otherwise produced from any suitable material or combination of materials, including polymeric materials, composite materials, metallic materials, ceramic materials, or combinations thereof. The peripheral corner regions of the top cover assembly 104 are shown with a smooth and continuous profile, although the peripheral corner regions may, in other embodiments, include one or more chamfered portions, beveled portions, radiused portions, stepped portions, or other contour features. The particular geometry of the top cover assembly 104 shown in FIG. 2 is illustrative and not limiting.

### Upper Housing

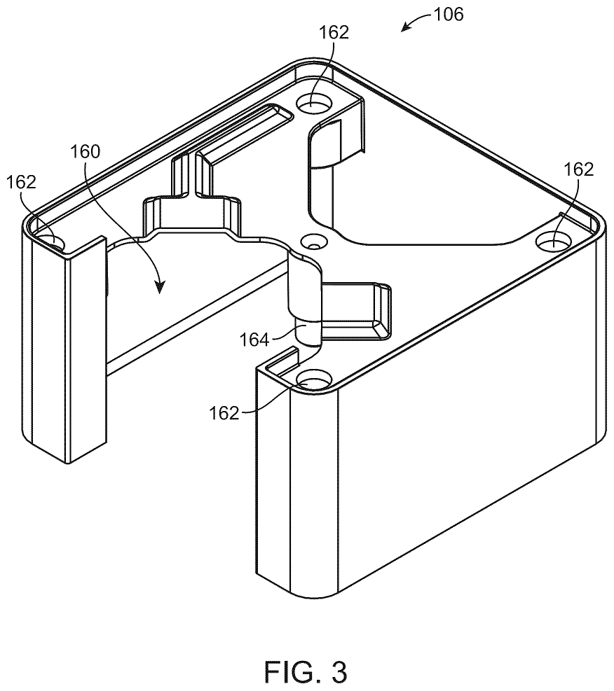

FIG. 3 illustrates a perspective view of the upper housing 106 of the medication storage and dispensing apparatus 100. As described with reference to FIG. 1A and FIG. 1B, the upper housing 106 is disposed below the top cover assembly 104 and above the lower housing 108 in the assembled state of FIG. 1A and defines the upper structural region of the apparatus 100. In FIG. 3, the upper housing 106 is shown as a discrete component, thereby exposing an interior of the upper housing 106.

In the illustrated embodiment, the upper housing 106 has a generally rectilinear body with a generally rectangular peripheral profile and rounded vertical corner regions, the upper housing 106 defining the upper interior volume sized to receive the support structure 134, the rotatable storage member 132, and the movable cover 114 therewithin as previously described with reference to FIG. 1B. The upper housing 106 includes the side wall portions and the front wall portions as previously described with reference to FIG. 1A, extending generally vertically about the upper interior volume.

An open front region 160 is defined at a front portion of the upper housing 106. In the illustrated embodiment, the open front region 160 interrupts the front wall portions of the upper housing 106 and extends inwardly from a front edge region of the upper housing 106. The open front region 160 has a generally curved inner boundary corresponding generally to the curved inner boundary of the cutout 112 of the top cover assembly 104. In the assembled state of FIG. 1A, the open front region 160 is positioned below the cutout 112 of the top cover assembly 104 such that the open front region 160 and the cutout 112 cooperate to expose a portion of the movable cover 114 to a user from a front side of the apparatus 100. In other embodiments, the open front region 160 may have any other suitable shape, including a partially circular shape, a partially elliptical shape, a slot-like shape, a polygonal shape, or a compound profile, and may be sized to expose any selected portion of the movable cover 114 to user access through the cutout 112.

A plurality of mounting bores 162 is disposed on the upper housing 106 at spaced positions about the upper interior volume. In the illustrated embodiment, the plurality of mounting bores 162 includes a first mounting bore, a second mounting bore, and a third mounting bore disposed at spaced positions on an upper rim region of the upper housing 106, each mounting bore of the plurality of mounting bores 162 being configured to receive a corresponding fastener for retaining the upper housing 106 in a predetermined orientation relative to one or more adjacent components of the apparatus 100. In other embodiments, the plurality of mounting bores 162 may comprise two mounting bores, three mounting bores, four mounting bores, or any other number of mounting bores sufficient to retain the upper housing 106, and may comprise threaded bores, unthreaded bores, counterbored bores, countersunk bores, blind bores, through bores, or any combination thereof.

A depending support element 164 extends downwardly from an inner surface of an upper wall portion of the upper housing 106 within the upper interior volume. In some embodiments, the depending support element 164 extends from an inner surface of an upper wall portion of the upper housing 106. In other embodiments, the depending support element 164 extends from another wall portion of the housing, including, for example, the top cover assembly 104 or a wall portion of the lower housing 108. In the illustrated embodiment, the depending support element 164 projects a predetermined distance downwardly relative to the upper wall portion of the upper housing 106 and is arranged generally coaxially with the generally vertical axis. The depending support element 164 is configured to cooperate with an upper end of the rotatable storage member 132 to rotatably support the upper end of the rotatable storage member 132 for rotation about the generally vertical axis in the assembled state of FIG. 1A. In other embodiments, the depending support element 164 may comprise a depending boss, a depending post, a depending pin, a depending shaft, or any combination thereof, and may be integrally formed with or separately attached to the upper housing 106.

### Support Structure

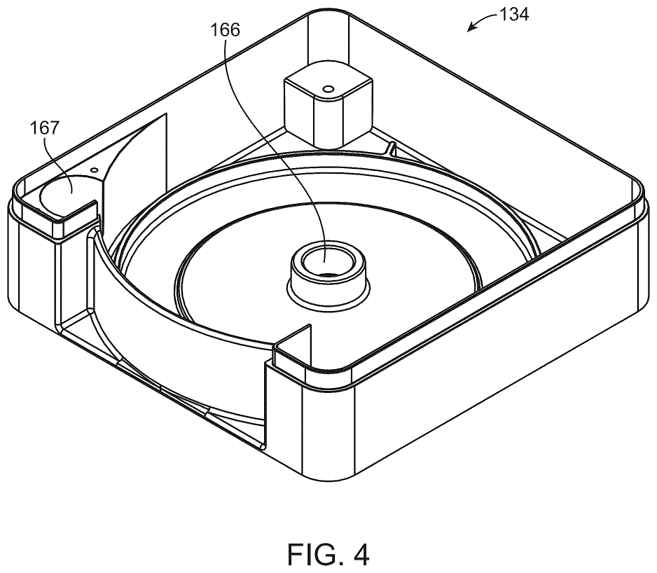

FIG. 4 illustrates a perspective view of the support structure 134 of the medication storage and dispensing apparatus 100. As described with reference to FIG. 1B, the support structure 134 is assembled with the upper housing 106 in the assembled state of FIG. 1A such that the support structure 134 and the upper housing 106 cooperate to define an interior space for receiving the rotatable storage member 132 therewithin. The support structure 134 does not rotate, while the rotatable storage member 132 is received by and rotatable relative to the support structure 134 about the generally vertical axis. In FIG. 4, the support structure 134 is shown as a discrete component, thereby exposing an interior of the support structure 134.

In the illustrated embodiment, the support structure 134 has a generally rectangular body with rounded vertical corner regions, the support structure 134 being sized to assemble with the upper housing 106 in the assembled state of FIG. 1A. The support structure 134 includes a generally cup-shaped or annular receiving region defined within the rectangular body, the cup-shaped or annular receiving region being configured to receive the rotatable storage member 132 from above as previously described with reference to FIG. 1B. A floor portion of the cup-shaped or annular receiving region includes a generally circular interior contour arranged generally concentrically about the generally vertical axis and configured to rotatably support the rotatable storage member 132 in the assembled state of FIG. 1A.

A central bushing 166 is disposed on the floor portion of the cup-shaped or annular receiving region of the support structure 134 and is arranged generally concentrically with the generally circular interior contour. In the illustrated embodiment, the central bushing 166 projects upwardly relative to the floor portion and defines a central through-passage arranged generally coaxially with the generally vertical axis. In the assembled state of FIG. 1A, the central bushing 166 is configured to receive a rotational bearing within the central through-passage of the central bushing 166, the rotational bearing rotatably supporting a central projecting element of the rotatable storage member 132 that extends downwardly through the central bushing 166. The central projecting element of the rotatable storage member 132 is coupled to the female coupler 142 by way of a fastener disposed at a lower end of the central projecting element, such that rotary drive imparted by engagement of the corresponding male coupler carried by the output shaft of the first actuator 146 with the female coupler 142 is transmitted through the fastener and the central projecting element to the rotatable storage member 132. In other embodiments, the central bushing 166 may comprise a unitary bushing, a multi-piece bushing, a metallic bushing, a polymeric bushing, or any combination thereof, and the rotational bearing received within the central bushing 166 may comprise a sleeve bearing, a ball bearing, a roller bearing, a journal bearing, a needle bearing, or any combination thereof.

An actuator-receiving recess 167 is defined at a peripheral region of the support structure 134. In the illustrated embodiment, the actuator-receiving recess 167 interrupts a peripheral wall of the support structure 134 and is configured to receive the second actuator 130 and the drive member 140 therewithin in the assembled state of FIG. 1A, thereby permitting placement of the second actuator 130 and the drive member 140 adjacent the support structure 134 within the upper interior volume of the upper housing 106. In other embodiments, the actuator-receiving recess 167 may comprise a notch, a cutout, a slot, or any combination thereof, and may be sized to accommodate any selected size and shape of the second actuator 130 and the drive member 140.

### Movable Cover

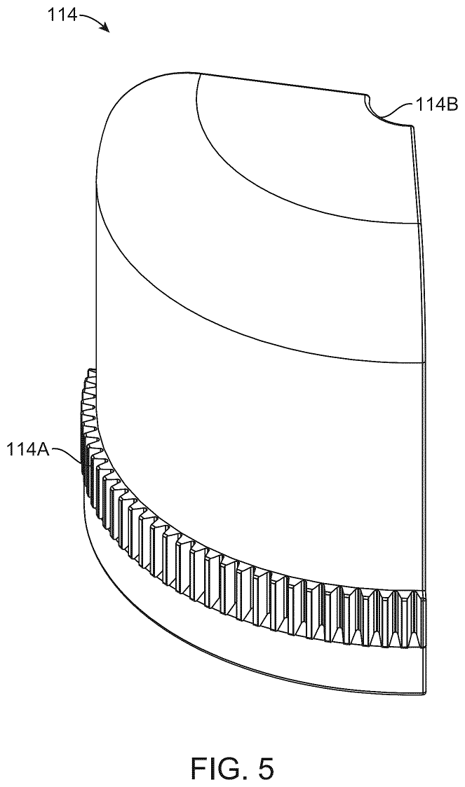

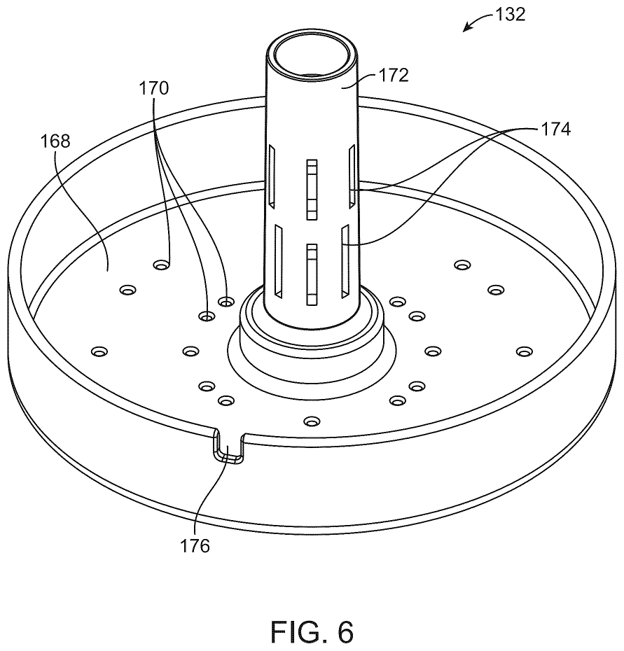

FIG. 5 illustrates a perspective view of the movable cover 114 of the medication storage and dispensing apparatus 100. As described with reference to FIG. 1A and FIG. 1B, the movable cover 114 is positioned within the housing 102 below the top cover assembly 104 and is exposed through the cutout 112 in the assembled state of FIG. 1A. In FIG. 5, the movable cover 114 is shown as a discrete component such that an outer wall of the movable cover 114 and the plurality of drive features 114A disposed on the outer wall are more fully visible than in FIG. 1A and FIG. 1B.

In the illustrated embodiment, the movable cover 114 has a generally partially cylindrical body disposed about a generally vertical axis. The movable cover 114 includes an upper portion having a generally smooth surface profile and a lower portion carrying the plurality of drive features 114A. An upper edge region of the movable cover 114 includes a recessed contour 114B configured to engage the depending support element 164 of the upper housing 106 in the assembled state of FIG. 1A. The depending support element 164 supports the movable cover 114 at the recessed contour 114B as the movable cover 114 is moved between a closed position and an open position by way of the drive member 140 driven by the second actuator 130. In some embodiments, the movable cover 114 may comprise a generally continuous cylindrical wall, a partial cylindrical wall, an arcuate panel, a multi-section assembly, or another rotatable closure geometry suitable for selectively obscuring and exposing an access region of the apparatus 100.

The plurality of drive features 114A is shown in FIG. 5 as a toothed region extending circumferentially along the lower portion of the outer wall of the movable cover 114. In the illustrated embodiment, the plurality of drive features 114A is configured to drivingly engage the drive member 140 driven by the second actuator 130 as described with reference to FIG. 1B. In the assembled state, motion imparted by the second actuator 130 to the drive member 140 is transmitted to the movable cover 114 through engagement of the drive member 140 with the plurality of drive features 114A, thereby moving the movable cover 114 between a closed position obscuring an access region of the apparatus 100 and an open position exposing the access region. In some embodiments, the plurality of drive features 114A comprises a plurality of gear teeth. In other embodiments, the plurality of drive features 114A may comprise ridges, friction-engaging surface features, magnetic drive features, or any combination thereof.

### Rotatable Storage Member

FIG. 6 illustrates a perspective view of the rotatable storage member 132 of the medication storage and dispensing apparatus 100. As described with reference to FIG. 1B and FIG. 4, the rotatable storage member 132 is received within the support structure 134 in the assembled state of FIG. 1A and is rotatable relative to the support structure 134 about the generally vertical axis to align a selected medication container 136 carried by the rotatable storage member 132 with the access region of the apparatus 100. In FIG. 6, the rotatable storage member 132 is shown as a discrete component such that a base plate 168 and a central pillar 172 of the rotatable storage member 132 are visible.

In the illustrated embodiment, the rotatable storage member 132 includes a base plate 168 having a generally circular plan profile arranged generally concentrically about the generally vertical axis. The base plate 168 includes a plurality of anchor positions 170 disposed circumferentially thereon at substantially regular angular intervals about the generally vertical axis. Each anchor position of the plurality of anchor positions 170 is configured to cooperate with a corresponding feature of a removable partition (not shown in FIG. 6) to removably retain the removable partition on the base plate 168 as previously described with reference to FIG. 1B. In other embodiments, the plurality of anchor positions 170 may comprise a plurality of through-bores, a plurality of blind bores, a plurality of recesses, a plurality of protrusions, a plurality of magnetic features, or any combination thereof, and may be arranged in a single circumferential array, a plurality of concentric circumferential arrays, or any other angularly distributed arrangement suitable for cooperating with the corresponding features of the removable partitions.

A central pillar 172 extends upwardly from the base plate 168 of the rotatable storage member 132. In the illustrated embodiment, the central pillar 172 has a generally cylindrical body arranged generally coaxially with the generally vertical axis and extends a predetermined distance upwardly relative to the base plate 168. The central pillar 172 includes a plurality of vertical cutouts 174 extending circumferentially about the central pillar 172 at substantially regular angular intervals about the generally vertical axis. Each vertical cutout of the plurality of vertical cutouts 174 is configured to cooperate with a corresponding feature of the removable partition to removably retain the removable partition between the base plate 168 and the central pillar 172 as previously described with reference to FIG. 1B. In other embodiments, the plurality of vertical cutouts 174 may comprise a plurality of vertical slots, a plurality of vertical grooves, a plurality of vertical channels, a plurality of recessed features, or any combination thereof, and may be arranged in any angularly distributed pattern suitable for cooperating with the corresponding features of the removable partitions.

An angular reference feature 176 is disposed on a periphery of the rotatable storage member 132. In the illustrated embodiment, the angular reference feature 176 has a generally protruding or recessed configuration and is positioned at a peripheral region of the base plate 168. In the assembled state of FIG. 1A, the angular reference feature 176 is configured to cooperate with a fixed reference structure fixedly mounted relative to the housing 102 to establish a home angular reference for the rotatable storage member 132. The fixed reference structure may be mounted on, formed with, or fixed relative to one or more of the housing 102, the upper housing 106, the support structure 134, the lighting floor 144, or another non-rotating structural component of the apparatus 100, and is not separately shown in FIG. 6. In some embodiments, cooperation between the angular reference feature 176 and the fixed reference structure may be detected mechanically, magnetically, optically, electrically, or by any other sensing arrangement suitable for establishing or verifying the home angular reference of the rotatable storage member 132. In other embodiments, the angular reference feature 176 may comprise a protrusion, a notch, a magnetic feature, an optical-marker feature, a hall-sensor target, or any combination thereof, and may be integrally formed with or separately attached to the rotatable storage member 132.

### Removable Partition

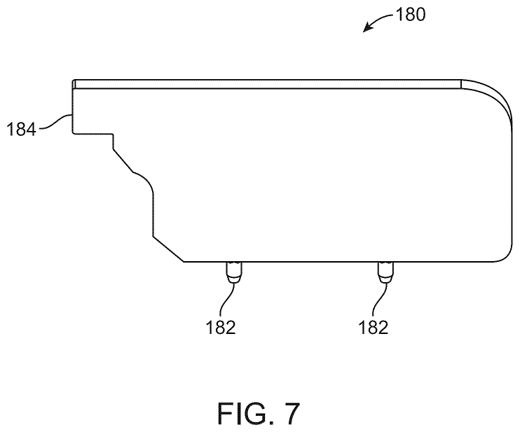

FIG. 7 illustrates a perspective view of a removable partition 180 of the medication storage and dispensing apparatus 100. As described with reference to FIG. 1B and FIG. 6, the removable partition 180 is removably engageable with the rotatable storage member 132 in the assembled state of FIG. 1A to define, in cooperation with the base plate 168 and the central pillar 172 of the rotatable storage member 132, a compartment configured to receive the medication container 136. In FIG. 7, the removable partition 180 is shown as a discrete component such that engagement features of the removable partition 180 are visible.

In the illustrated embodiment, the removable partition 180 includes a generally rectangular planar body and a plurality of engagement legs 182 extending downwardly from an operative lower edge of the generally rectangular planar body. The plurality of engagement legs 182 includes a first engagement leg and a second engagement leg disposed at spaced positions along the operative lower edge of the generally rectangular planar body. Each engagement leg of the plurality of engagement legs 182 is configured to engage a corresponding anchor position of the plurality of anchor positions 170 of the base plate 168 of the rotatable storage member 132 in the assembled state of FIG. 1A, thereby removably retaining the operative lower edge of the generally rectangular planar body on the base plate 168. In other embodiments, the generally rectangular planar body may comprise a curved body, a multi-section body, a folding body, or any other panel-like geometry suitable for subdividing a volume defined by the rotatable storage member 132 into compartments, and the plurality of engagement legs 182 may comprise two engagement legs, three engagement legs, four engagement legs, or any other number of engagement legs sufficient to removably retain the removable partition 180 on the base plate 168, and may comprise depending pegs, depending pins, depending tabs, snap-fit features, friction-fit features, or any combination thereof.

The removable partition 180 further includes a pillar-engagement edge 184 disposed along an operative inner side of the generally rectangular planar body. The pillar-engagement edge 184 has a stepped profile extending generally vertically along the operative inner side and is configured to engage a corresponding vertical cutout of the plurality of vertical cutouts 174 of the central pillar 172 of the rotatable storage member 132 in the assembled state of FIG. 1A, thereby removably retaining the pillar-engagement edge 184 within the corresponding vertical cutout of the central pillar 172. In other embodiments, the pillar-engagement edge 184 may comprise a generally vertical projecting tab, a generally vertical recessed channel, a tongue feature, a key feature, a snap-fit feature, a friction-fit feature, or any combination thereof.

### Female Coupler and Male Coupler

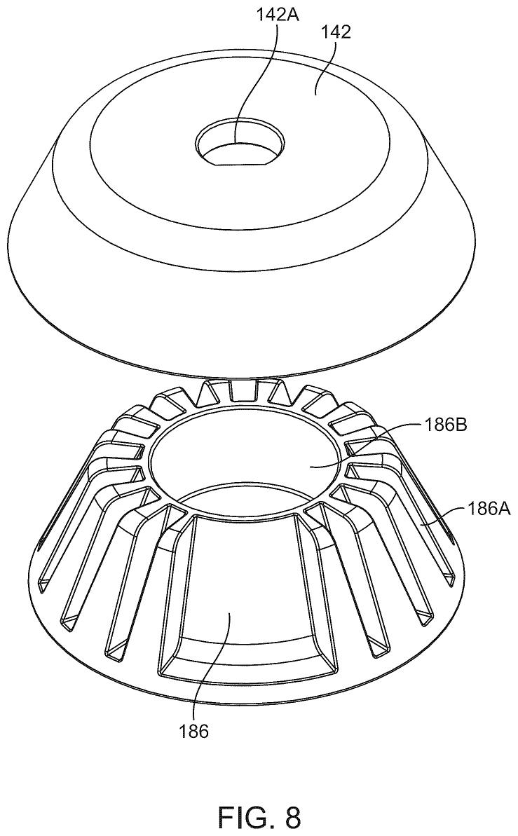

FIG. 8 illustrates a perspective view of the female coupler 142 and a male coupler 186 of the medication storage and dispensing apparatus 100. As described with reference to FIG. 1B, the female coupler 142 is disposed on the underside region of the rotatable storage member 132 and the male coupler 186 is carried by the output shaft of the first actuator 146. In the assembled state of FIG. 1A, the female coupler 142 receives the male coupler 186 from below, and rotary drive imparted by the first actuator 146 to the male coupler 186 is transmitted through the female coupler 142 to the rotatable storage member 132. In FIG. 8, the female coupler 142 and the male coupler 186 are shown as discrete components in vertically separated relation to expose engagement features of the female coupler 142 and the male coupler 186.

In the illustrated embodiment, the female coupler 142 has a generally dome-shaped body defining an operative upper surface and an operative inner cavity, the operative inner cavity being defined on an operative underside of the generally dome-shaped body and configured to receive the male coupler 186 from below in the assembled state of FIG. 1A. The operative upper surface of the generally dome-shaped body further defines a central opening 142A. In other embodiments, the generally dome-shaped body of the female coupler 142 may comprise a generally cup-shaped body, a generally annular body, a generally cylindrical body, or any other body geometry suitable for receiving the male coupler 186 to transmit rotary drive.

In the illustrated embodiment, the male coupler 186 has a generally frusto-conical body and includes a plurality of circumferential engagement features 186A disposed on an outer surface of the generally frusto-conical body and a central opening 186B defined at an upper end of the generally frusto-conical body. The plurality of circumferential engagement features 186A extends generally vertically along the outer surface of the generally frusto-conical body at substantially regular angular intervals about the generally vertical axis. Each engagement feature of the plurality of circumferential engagement features 186A is configured to engage a corresponding engagement feature of the operative inner cavity of the female coupler 142 in the assembled state of FIG. 1A, thereby providing the keyed engagement between the male coupler 186 and the female coupler 142 that re-establishes the predetermined angular relationship between the female coupler 142 and the male coupler 186 upon each disassembly and reassembly of the rotatable storage member 132 from the housing 102, without requiring a user-administered calibration procedure. In other embodiments, the plurality of circumferential engagement features 186A may comprise a plurality of teeth, a plurality of splines, a plurality of keys, a plurality of dogs, a plurality of polygonal facets, or any combination thereof.

### First Actuator

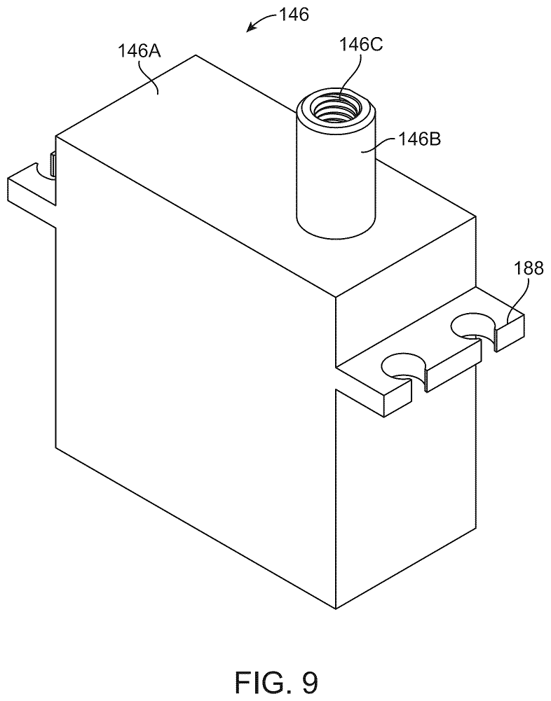

FIG. 9 illustrates a perspective view of the first actuator 146 of the medication storage and dispensing apparatus 100. As described with reference to FIG. 1B, the first actuator 146 is supported within the lower housing 108 in the assembled state of FIG. 1A and is configured to impart rotation to the rotatable storage member 132 through the male coupler 186 and the female coupler 142 about the generally vertical axis. In FIG. 9, the first actuator 146 is shown as a discrete component such that an actuator body 146A, an output shaft 146B, and a plurality of mounting tabs 188 of the first actuator 146 are visible.

In the illustrated embodiment, the actuator body 146A has a generally rectangular configuration with an upper surface, a lower surface, and a plurality of side surfaces extending between the upper surface and the lower surface. The actuator body 146A houses electromechanical components (not visible in FIG. 9) configured to impart rotational motion to the output shaft 146B in response to electrical signals received from a controller of the apparatus 100. In other embodiments, the actuator body 146A may have a generally cubic configuration, a generally cylindrical configuration, a generally polygonal configuration, or any other configuration suitable for housing the electromechanical components.

The output shaft 146B extends upwardly from the upper surface of the actuator body 146A and is arranged generally coaxially with the generally vertical axis in the assembled state of FIG. 1A. In the illustrated embodiment, the output shaft 146B has a generally cylindrical body and defines a central engagement feature 146C at an upper end of the output shaft 146B, the central engagement feature 146C being configured to receive and retain the male coupler 186 such that rotation imparted by the first actuator 146 to the output shaft 146B is transmitted to the male coupler 186 and, through the male coupler 186, to the female coupler 142 and the rotatable storage member 132. In some embodiments, the central engagement feature 146C comprises a threaded recess configured to receive a corresponding fastener of the male coupler 186. In other embodiments, the central engagement feature 146C may comprise a keyed recess, a splined recess, a polygonal recess, a press-fit interface, an interference-fit interface, or any combination thereof.

The plurality of mounting tabs 188 extends laterally from one or more side surfaces of the actuator body 146A. In the illustrated embodiment, the plurality of mounting tabs 188 includes a first mounting tab and a second mounting tab disposed at spaced positions along one of the side surfaces of the actuator body 146A, each mounting tab of the plurality of mounting tabs 188 including a mounting opening configured to receive a corresponding fastener for retaining the first actuator 146 in a predetermined orientation relative to the lower housing 108 in the assembled state of FIG. 1A. In other embodiments, the plurality of mounting tabs 188 may comprise two mounting tabs, three mounting tabs, four mounting tabs, or any other number of mounting tabs sufficient to retain the first actuator 146 relative to the lower housing 108, and may comprise flange features, bracket features, snap-fit features, or any combination thereof.

### Drive Transmission From First Actuator to Rotatable Storage Member

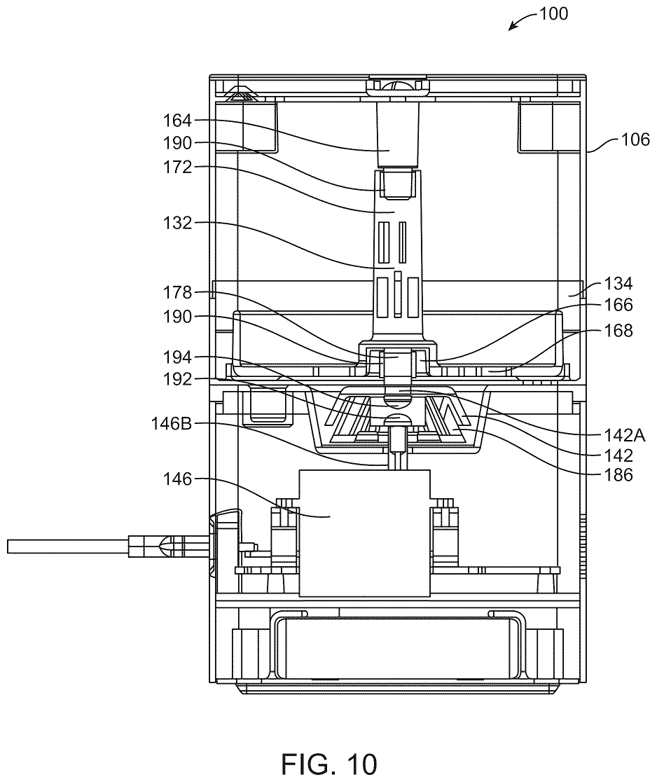

FIG. 10 illustrates a sectional view of a drive transmission arrangement of the medication storage and dispensing apparatus 100. In FIG. 10, the rotatable storage member 132, the support structure 134, the central bushing 166, the upper housing 106, the female coupler 142, the male coupler 186, the first actuator 146, and the output shaft 146B of the first actuator 146, all as previously described with reference to FIG. 1B, FIG. 3, FIG. 4, FIG. 6, FIG. 8, and FIG. 9, are shown in cross-section to expose internal features and engagement relationships involved in transmitting rotary drive from the first actuator 146 to the rotatable storage member 132.

A central projecting element 178 of the rotatable storage member 132 extends downwardly from the base plate 168 of the rotatable storage member 132. In the illustrated embodiment, the central projecting element 178 has a generally elongated body arranged generally coaxially with the generally vertical axis and extends through the central bushing 166 of the support structure 134 in the assembled state of FIG. 1A.

The rotatable storage member 132 is rotatably supported about the generally vertical axis by a plurality of rotational bearings 190 cooperating with the central projecting element 178 and the central pillar 172 of the rotatable storage member 132. In the illustrated embodiment, the plurality of rotational bearings 190 includes a first rotational bearing disposed within the central through-passage of the central bushing 166 and a second rotational bearing disposed at an operative top end of the central pillar 172. The first rotational bearing rotatably supports the central projecting element 178 within the central bushing 166 for rotation about the generally vertical axis, and the second rotational bearing is interposed between the operative top end of the central pillar 172 and the depending support element 164 of the upper housing 106 as described with reference to FIG. 3 to rotatably support the operative top end of the central pillar 172 relative to the depending support element 164 for rotation about the generally vertical axis. In other embodiments, each rotational bearing of the plurality of rotational bearings 190 may comprise a sleeve bearing, a ball bearing, a roller bearing, a journal bearing, a needle bearing, a polymeric bearing, a metallic bearing, a self-lubricating bearing, or any combination thereof.

The female coupler 142 is coupled to the central projecting element 178 of the rotatable storage member 132 by a second fastener 194 disposed between the female coupler 142 and a lower end of the central projecting element 178. In the illustrated embodiment, the second fastener 194 extends through the central opening 142A of the female coupler 142 as described with reference to FIG. 8 and engages the lower end of the central projecting element 178, thereby rigidly coupling the female coupler 142 to the central projecting element 178 such that rotation of the female coupler 142 about the generally vertical axis is directly transmitted through the second fastener 194 and the central projecting element 178 to the rotatable storage member 132. In other embodiments, the second fastener 194 may comprise a machine screw, a threaded bolt, a self-tapping screw, a captive fastener, a press-fit pin, or any combination thereof.

The male coupler 186 is coupled to the output shaft 146B of the first actuator 146 by a first fastener 192 disposed between the male coupler 186 and an upper end of the output shaft 146B. In the illustrated embodiment, the first fastener 192 extends through the central opening 186B of the male coupler 186 as described with reference to FIG. 8 and engages the central engagement feature 146C of the output shaft 146B as described with reference to FIG. 9, thereby rigidly coupling the male coupler 186 to the output shaft 146B such that rotation of the output shaft 146B about the generally vertical axis is directly transmitted through the first fastener 192 to the male coupler 186. In other embodiments, the first fastener 192 may comprise a machine screw, a threaded bolt, a self-tapping screw, a captive fastener, a press-fit pin, or any combination thereof.

In the illustrated embodiment, the male coupler 186 is received within the operative inner cavity of the female coupler 142 such that the plurality of circumferential engagement features 186A of the male coupler 186 engages a corresponding plurality of engagement features defined within the operative inner cavity of the female coupler 142 as described with reference to FIG. 8, thereby providing the keyed engagement between the male coupler 186 and the female coupler 142 that re-establishes the predetermined angular relationship between the female coupler 142 and the male coupler 186 upon each disassembly and reassembly of the rotatable storage member 132 from the housing 102. Accordingly, rotary drive imparted by the first actuator 146 to the output shaft 146B is transmitted through the first fastener 192 to the male coupler 186, through the keyed engagement between the male coupler 186 and the female coupler 142 to the female coupler 142, through the second fastener 194 to the central projecting element 178, and through the central projecting element 178 to the rotatable storage member 132, thereby rotating the rotatable storage member 132 about the generally vertical axis to align a selected medication container 136 with the access region of the apparatus 100.

Accordingly, FIG. 10 illustrates the drive transmission arrangement of the medication storage and dispensing apparatus 100 in sectional view and shows, by way of example, the rotatable storage member 132 with the central pillar 172 and the central projecting element 178, the depending support element 164 of the upper housing 106, the central bushing 166 of the support structure 134, the plurality of rotational bearings 190 cooperating with the central projecting element 178 and the central pillar 172, the female coupler 142 with the central opening 142A, the male coupler 186 with the plurality of circumferential engagement features 186A and the central opening 186B, the first fastener 192 coupling the male coupler 186 to the output shaft 146B of the first actuator 146, and the second fastener 194 coupling the female coupler 142 to the central projecting element 178. The embodiment shown in FIG. 10 is exemplary only, and the relative shape, proportions, and engagement relationships of the drive transmission arrangement may be varied in other embodiments without departing from the scope of the disclosed technology.

### Second Actuator

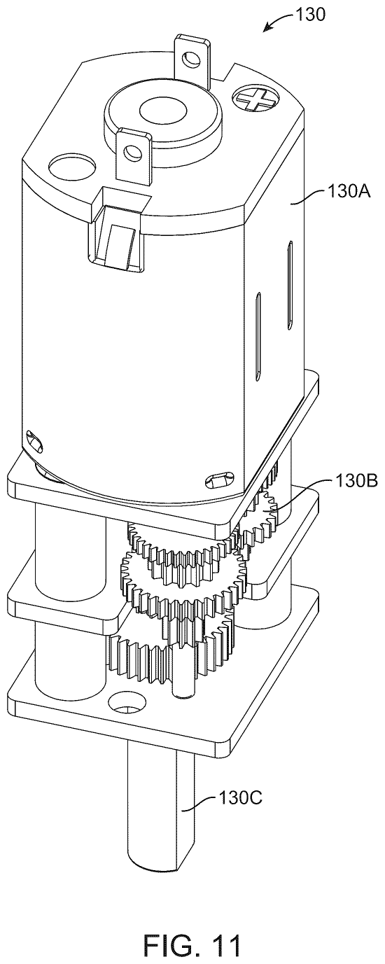

FIG. 11 illustrates a perspective view of the second actuator 130 of the medication storage and dispensing apparatus 100. As described with reference to FIG. 1B, the second actuator 130 is mechanically coupled to the movable cover 114 by way of the drive member 140 and is configured to move the movable cover 114 between the closed position and the open position in the assembled state of FIG. 1A. In FIG. 11, the second actuator 130 is shown as a discrete component including an actuator body 130A having a generally cylindrical configuration, a gear arrangement 130B disposed adjacent the actuator body 130A and configured to transmit and reduce rotational motion received from the actuator body 130A, and an output shaft 130C extending from the gear arrangement 130B and configured to drivingly engage the drive member 140 in the assembled state of FIG. 1A. In other embodiments, the second actuator 130 may comprise a stepper motor, a servo motor, a geared motor, a brushless motor, a linear actuator coupled to a rotary mechanism, a solenoid-driven actuator, or another electromechanical drive element suitable for moving the movable cover 114, and the gear arrangement 130B may comprise a planetary gear arrangement, a spur gear arrangement, a worm gear arrangement, a helical gear arrangement, or any combination thereof.

In the illustrated embodiment, the drive member 140 includes a plurality of engagement features arranged on a peripheral surface of the drive member 140. The plurality of engagement features of the drive member 140 is complementary to and meshingly engaged with the plurality of drive features 114A disposed on the outer wall of the movable cover 114 such that rotational motion imparted by the second actuator 130 to the drive member 140 is transmitted through the meshing engagement between the plurality of engagement features of the drive member 140 and the plurality of drive features 114A of the movable cover 114 to the movable cover 114, thereby driving the movable cover 114 about the generally vertical axis between the closed position and the open position. In some embodiments, the plurality of engagement features of the drive member 140 comprises a plurality of gear teeth and the plurality of drive features 114A of the movable cover 114 comprises a corresponding plurality of gear teeth, the two pluralities of gear teeth cooperatively defining a gear pair through which rotational motion of the drive member 140 is transmitted to the movable cover 114. In other embodiments, the plurality of engagement features of the drive member 140 and the plurality of drive features 114A of the movable cover 114 may comprise complementary frictional surfaces, complementary magnetic features, complementary belt-engaging features, or other complementary engagement features suitable for transmitting rotational motion from the drive member 140 to the movable cover 114. The particular form of the meshing engagement between the plurality of engagement features of the drive member 140 and the plurality of drive features 114A of the movable cover 114 is illustrative and not limiting.

### Lighting Floor

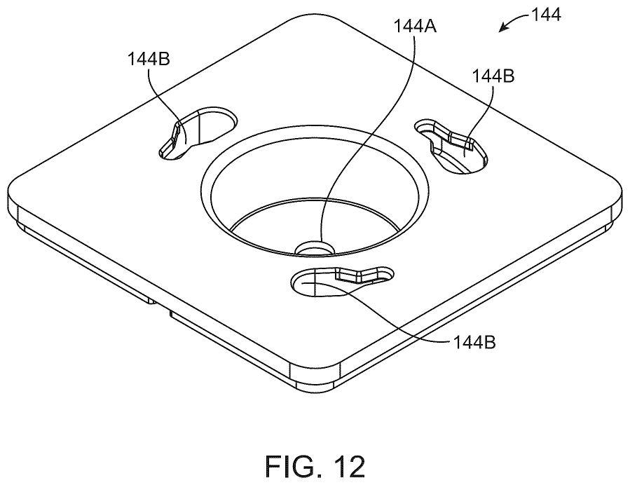

FIG. 12 illustrates a perspective view of the lighting floor 144 of the medication storage and dispensing apparatus 100. As described with reference to FIG. 1B, the lighting floor 144 is positioned between the support structure 134 above and the lower housing 108 below in the assembled state of FIG. 1A. In FIG. 12, the lighting floor 144 is shown as a discrete component including a generally plate-like body, the central aperture 144A aligned generally with the generally vertical axis and configured to receive the output shaft 146B of the first actuator 146 therethrough, and a plurality of mounting features 144B arranged at spaced positions about the central aperture 144A and configured to receive corresponding fasteners for retaining the lighting floor 144 in a predetermined orientation relative to one or more of the support structure 134 and the lower housing 108. In some embodiments, the lighting floor 144 comprises a translucent material and further includes one or more light sources arranged internally of the lighting floor 144 and configured to illuminate compartments of the rotatable storage member 132 from below through the translucent material of the lighting floor 144. In other embodiments, the plurality of mounting features 144B may comprise keyhole slots, mounting bores, snap-fit features, alignment posts, friction-fit features, or any combination thereof.

### Lower Housing

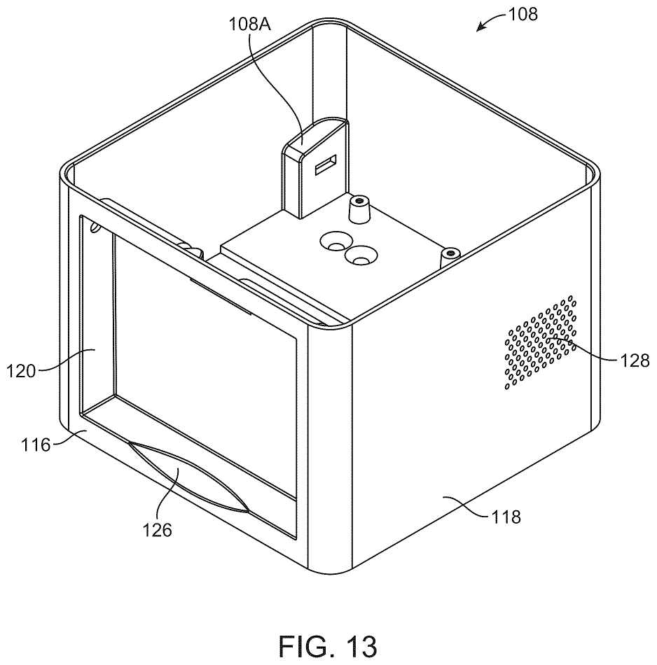

FIG. 13 illustrates a perspective view of the lower housing 108 of the medication storage and dispensing apparatus 100. As described with reference to FIG. 1A and FIG. 1B, the lower housing 108 is disposed below the upper housing 106 in the assembled state of FIG. 1A and defines the lower portion of the apparatus 100. The lower housing 108 carries the front face 116 with the opening 120 receiving the user-interface display 122 (the user-interface display 122 being shown in FIGS. 1A and 1B), the side face 118 with the plurality of perforations 128, and the recess 126 at the lower-front region as previously described with reference to FIG. 1A. In FIG. 13, the lower housing 108 is shown as a discrete component with an open top, thereby exposing the interior volume of the lower housing 108.

In the illustrated embodiment, the lower housing 108 has a generally box-shaped body with rounded vertical corner regions and a precision-molded configuration. The interior volume is configured to function as a core electronics housing of the apparatus 100 and is sized to receive the first actuator 146 and one or more electrical components of the apparatus 100, including a controller, power-management circuitry, battery management circuitry, and one or more wiring harnesses (not visible in FIG. 13), in a compact arrangement. The first actuator 146 is configured to be mounted within the interior volume such that the output shaft 146B of the first actuator 146 extends generally vertically upwardly to drivingly engage the male coupler 186 in the assembled state of FIG. 1A.

The controller, the non-transitory storage medium, the network interface, the power-management circuitry, the battery management circuitry, and the one or more wiring harnesses may be received within the interior volume of the lower housing 108, but are not separately shown in FIG. 13. In some embodiments, the controller is operatively coupled to the first actuator 146, the second actuator 130, the plurality of force sensors 150, the user-interface display 122, the non-transitory storage medium, and the network interface. The non-transitory storage medium may store one or more of a medication schedule, an installed configuration of the rotatable storage member 132, expected weight-delta values, dispensing-event records, verification results, alert conditions, and computer-executable instructions for operating the apparatus 100. The network interface may be configured to transmit an audit record, a verification failure alert, a dispensing status, or another operational notification to a remote system.

A connector receptacle 108A is disposed within the interior volume of the lower housing 108 at a peripheral region of the interior volume. In the illustrated embodiment, the connector receptacle 108A has a generally upstanding configuration including an opening on a face thereof and is configured to receive a corresponding external connector for electrical communication with the electrical components received within the interior volume of the lower housing 108. In some embodiments, the connector receptacle 108A may comprise a USB-C connector receptacle, a USB-A connector receptacle, a barrel-jack connector receptacle, an inductive charging interface, or any combination thereof.

The plurality of perforations 128 disposed on the side face 118 of the lower housing 108 is configured to provide passive thermal ventilation and acoustic transmission between the interior volume of the lower housing 108 and an exterior environment. In some embodiments, additional perforations may be disposed on a lower wall portion of the lower housing 108 to further facilitate the passive thermal ventilation and the acoustic transmission for one or more acoustic components of the apparatus 100, including a microphone, a speaker, or both. The lower housing 108 is further configured to assemble with the lighting floor 144 in the assembled state of FIG. 1A such that the lighting floor 144 is positioned above an open upper region of the lower housing 108 as described with reference to FIG. 12.

### Actuator Mounting Bracket

FIG. 14 illustrates a perspective view of the actuator mounting bracket 148 of the medication storage and dispensing apparatus 100. As described with reference to FIG. 1B and FIG. 13, the first actuator 146 is supported within the interior volume of the lower housing 108 in the assembled state of FIG. 1A. In the illustrated embodiment, the actuator mounting bracket 148 is disposed within the interior volume of the lower housing 108 and is configured to receive and retain the first actuator 146 in a predetermined orientation relative to the lower housing 108. In FIG. 14, the actuator mounting bracket 148 is shown as a discrete component.

In the illustrated embodiment, the actuator mounting bracket 148 includes a generally planar base plate having a plurality of mounting bores 148A arranged at spaced positions about the periphery of the generally planar base plate and configured to receive corresponding fasteners for securing the actuator mounting bracket 148 to the lower housing 108 in the assembled state of FIG. 1A. A pair of upstanding bracket portions 148B extends upwardly from the generally planar base plate at spaced positions thereon. The pair of upstanding bracket portions 148B cooperates to define a receiving region between the pair of upstanding bracket portions 148B configured to receive and retain the first actuator 146 in a predetermined orientation such that the output shaft 146B of the first actuator 146 is positioned generally coaxially with the generally vertical axis of the apparatus 100 in the assembled state of FIG. 1A.

In the illustrated embodiment, each upstanding bracket portion of the pair of upstanding bracket portions 148B includes one or more motor-securing bores configured to align with the plurality of mounting tabs 188 of the first actuator 146 as described with reference to FIG. 9 and to receive corresponding fasteners for securing the first actuator 146 to the pair of upstanding bracket portions 148B. In some embodiments, the corresponding fasteners comprise torque-specified fasteners configured to retain the first actuator 146 to the pair of upstanding bracket portions 148B with a predetermined torque that resists loosening under vibration during operation of the first actuator 146.

In the illustrated embodiment, the actuator mounting bracket 148 is configured to distribute reaction forces from operation of the first actuator 146 across the interior volume of the lower housing 108 to reduce concentration of stress on any single mounting point of the lower housing 108. The actuator mounting bracket 148 may further include one or more clearance channels configured to permit routing of one or more wiring harnesses associated with the first actuator 146 from the actuator mounting bracket 148 to other electrical components of the apparatus 100. In some embodiments, the actuator mounting bracket 148 includes one or more anti-vibration features arranged at an interface between the actuator mounting bracket 148 and the first actuator 146 to reduce acoustic transmission from operation of the first actuator 146 to the lower housing 108. In other embodiments, the actuator mounting bracket 148 may comprise a unitary precision-machined body, a fabricated multi-piece assembly, a molded body, a cast body, an additively manufactured body, or any combination thereof, and may comprise polymeric materials, metallic materials, composite materials, or any combination thereof.

### Base

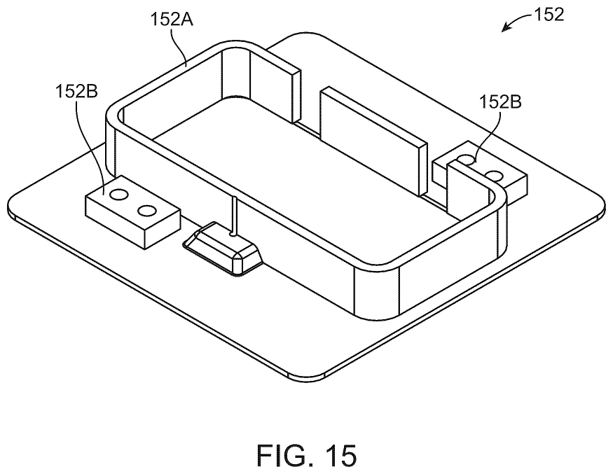

FIG. 15 illustrates a perspective view of the base 152 of the medication storage and dispensing apparatus 100. As described with reference to FIG. 1B, the base 152 forms a lower boundary of the apparatus 100 and supports the apparatus 100 in the assembled state of FIG. 1A. In FIG. 15, the base 152 is shown as a discrete component such that an upper surface of the base 152 and one or more features disposed thereon are visible.

In the illustrated embodiment, the base 152 has a generally planar body with the upper surface and a lower surface. The lower surface of the base 152 is configured to seat upon a horizontal support surface in the assembled state of FIG. 1A. The upper surface of the base 152 includes a battery-receiving region 152A defined by one or more upstanding walls extending upwardly from the upper surface of the base 152. The battery-receiving region 152A is configured to receive the battery 154 therein in the assembled state of FIG. 1A. In some embodiments, the battery 154 is securable to the battery-receiving region 152A by way of one or more captive screws extending from the lower surface of the base 152, the one or more captive screws being accessible for service from the lower surface of the base 152 to permit removal and replacement of the battery 154 without disassembly of the apparatus 100 from above.

The base 152 further includes one or more mounting features 152B disposed on the upper surface of the base 152 at spaced positions thereon. In the illustrated embodiment, the one or more mounting features 152B include a mounting block disposed at a peripheral region of the upper surface and an upstanding feature disposed adjacent the battery-receiving region 152A, the one or more mounting features 152B being configured to cooperate with one or more components of the apparatus 100 in the assembled state of FIG. 1A. In other embodiments, the one or more mounting features 152B may comprise mounting bosses, threaded bores, alignment posts, snap-fit features, sensor mounts, switch mounts, terminal contacts, or any combination thereof.

The upper surface of the base 152 is configured to receive the plurality of force sensors 150 thereon at angularly distributed positions about the generally vertical axis of the apparatus 100. In the assembled state of FIG. 1A, the plurality of force sensors 150 is mechanically interposed between the upper surface of the base 152 and one or more components of the apparatus 100 supported above the base 152, the plurality of force sensors 150 being configured to detect a vertical load transmitted from the one or more components to the base 152. The battery 154 received within the battery-receiving region 152A is mechanically supported by the base 152 such that the battery 154 is at least partially isolated from the vertical load detected by the plurality of force sensors 150, thereby permitting the plurality of force sensors 150 to detect changes in the vertical load associated with removal of medication containers from the apparatus 100 independently of a weight of the battery 154.

In other embodiments, the base 152 may comprise a unitary molded body, a fabricated assembly, a plate-and-frame structure, a footed structure including a plurality of feet disposed on the lower surface, or another support structure configured to seat upon a horizontal support surface and to support the apparatus 100.

In the assembled state of the apparatus 100, a vertical load corresponding to a sum of the weights of one or more components of the apparatus 100 supported above the plurality of force sensors 150 is transmitted downwardly through a structural load path of the apparatus 100 to the plurality of force sensors 150 disposed on the upper surface of the base 152 at angularly distributed positions about the generally vertical axis. The one or more components of the apparatus 100 supported above the plurality of force sensors 150 include the lower housing 108, the upper housing 106, the top cover assembly 104, the support structure 134, the rotatable storage member 132, the movable cover 114, the first actuator 146, the second actuator 130, the actuator mounting bracket 148, the lighting floor 144, and any medication containers 136 carried by the rotatable storage member 132. The battery 154 is received within the battery-receiving region 152A defined on the base 152 by the one or more upstanding walls extending upwardly from the upper surface of the base 152 and is thereby structurally isolated from the structural load path through the plurality of force sensors 150, such that the weight of the battery 154 does not contribute to the vertical load detected by the plurality of force sensors 150. The plurality of force sensors 150 is configured to detect the vertical load so transmitted thereto, the detected vertical load corresponding to the sum of the weights of the components of the apparatus 100 supported above the plurality of force sensors 150 at a given time. A change in the detected vertical load corresponds to a change in the weight of the one or more components supported above the plurality of force sensors 150, including, for example, removal or replacement of a medication container 136 of the plurality of medication containers 136 carried by the rotatable storage member 132 from or to the rotatable storage member 132 through the access region of the apparatus 100. The plurality of force sensors 150 thereby enables the apparatus 100 to detect a weight delta corresponding to removal of a selected medication container 136 from the rotatable storage member 132.

### Method of Dispensing Medication

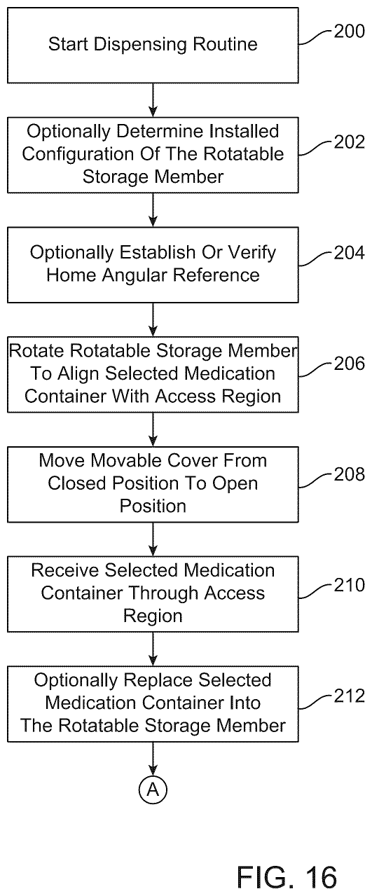

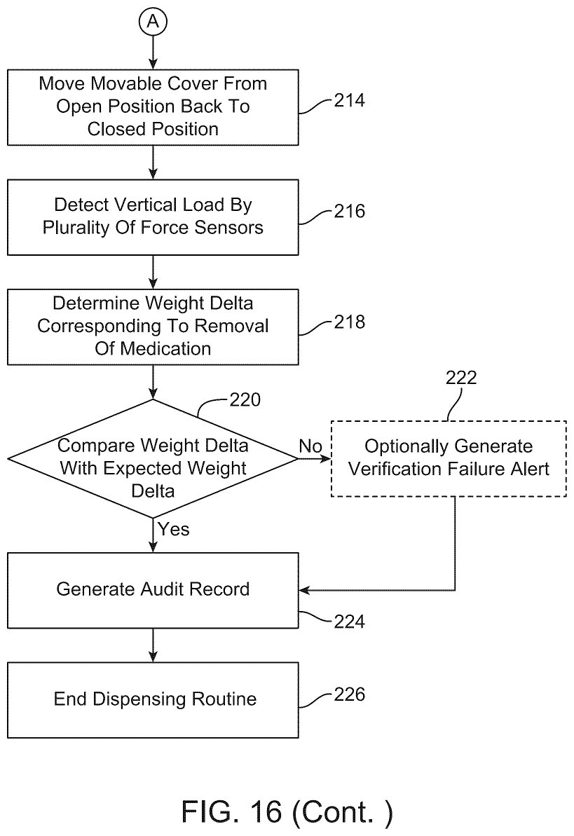

FIG. 16 illustrates a flow diagram of a method of dispensing medication using the medication storage and dispensing apparatus 100, according to certain embodiments. In the illustrated embodiment, the method of FIG. 16 may be carried out by coordinated operation of a controller of the apparatus 100, the first actuator 146, the second actuator 130, the rotatable storage member 132, the movable cover 114, the configuration-encoding interface of the apparatus, an angular reference feature of the rotatable storage member 132, a fixed reference structure fixedly mounted relative to the housing 102 or the support structure 134, the plurality of force sensors 150, and, in certain implementations, a non-transitory storage medium operatively coupled to the controller and a network interface communicatively coupled to the controller. The sequence shown in FIG. 16 is illustrative of one implementation and is not limiting unless expressly recited. Optional steps are shown in dashed-line boxes.

At step 200, a dispensing routine is started. In the illustrated embodiment, step 200 corresponds to initiation of a dispensing event under control of the controller. The controller may initiate the dispensing routine in response to expiration of a regimen schedule stored on a non-transitory storage medium, receipt of a user input, receipt of a caregiver command, receipt of a remote instruction, or another triggering event. Step 200 therefore corresponds to commencement of a controlled dispensing sequence for a selected medication container 136.

At optional step 202, the controller determines an installed configuration of the rotatable storage member 132. In the illustrated embodiment, step 202 is performed by reading a configuration-encoding interface physically encoding the installed configuration of the rotatable storage member 132. The controller may determine, based solely on the physical encoding of the configuration-encoding interface, whether the rotatable storage member 132 is presently arranged in a first configuration, a second configuration, or another supported compartment configuration. In some embodiments, the controller uses the determined installed configuration to configure dispensing logic, medication-position mapping, or schedule logic for the dispensing routine. Step 202 may be performed at startup, after reconfiguration of one or more removable partitions, after reassembly, or at another appropriate time.

At optional step 204, the controller establishes or verifies a home angular reference for the rotatable storage member 132. In the illustrated embodiment, step 204 is performed by operating the first actuator 146 to rotate the rotatable storage member 132 while monitoring for a reference condition defined by cooperation between an angular reference feature of the rotatable storage member 132 and a fixed reference structure fixedly mounted relative to the housing 102 or the support structure 134. In some embodiments, the controller performs a homing routine upon power-up or following disassembly and reassembly of the rotatable storage member 132. In other embodiments, the controller performs an angular verification operation to confirm that the rotatable storage member 132 is already in a known angular position. Step 204 therefore provides controlled angular referencing before a dispensing position is selected.

At step 206, the controller causes the first actuator 146 to rotate the rotatable storage member 132 to align a selected medication container 136 with an access region of the apparatus 100. In the illustrated embodiment, rotation of the first actuator 146 is transmitted through a corresponding male coupler and the female coupler 142 to the rotatable storage member 132, thereby rotating the rotatable storage member 132 about a generally vertical axis. The controller may determine a target angular position based on the selected medication, the installed configuration of the rotatable storage member 132, and a stored medication-position mapping. Step 206 therefore positions the selected medication container 136 at a dispensing location corresponding to the access region.

At step 208, the controller causes the second actuator 130 to move the movable cover 114 from a closed position to an open position. In the illustrated embodiment, the second actuator 130 drives the drive member 140, and the drive member 140 engages the plurality of drive features 114A disposed on the outer wall of the movable cover 114 to move the movable cover 114. In the closed position, the movable cover 114 obscures the access region. In the open position, the movable cover 114 exposes the selected medication container 136 aligned at step 206 while continuing to block access to non-selected medication containers. Step 208 therefore provides controlled access to the selected medication container 136.

At step 210, the selected medication container 136 is received through the access region. In the illustrated embodiment, step 210 corresponds to physical receipt, withdrawal, grasping, or removal of the selected medication container 136 by a user through the access region exposed by the movable cover 114. In some embodiments, the user may open the child-resistant cap 138, remove medication from the medication container 136, and thereafter re-close the child-resistant cap 138 before returning the medication container 136 to the apparatus 100. Step 210 therefore corresponds to the medication-access phase of the dispensing routine. In some embodiments, step 210 includes manual receipt of the selected medication container by a user, opening of the child-resistant cap, removal of medication, and replacement of the medication container into the apparatus. In other embodiments, the apparatus is configured to automatically dispense medication from the selected medication container after alignment of the selected medication container with the access region, such that manual opening of the medication container and manual removal of medication by the user are reduced or eliminated. In such embodiments, one or more dispensing operations may be performed under control of the controller before the movable cover is returned to the closed position, and the force-sensor-based verification and audit operations described herein may thereafter be performed in the same or similar manner.

In further embodiments, the apparatus 100 includes an automated dispensing mechanism operatively associated with the selected medication container 136 and controlled by the controller. The automated dispensing mechanism may be configured to dispense one or more medication units from the selected medication container 136 after alignment of the selected medication container 136 with the access region, with or without complete removal of the selected medication container 136 from the rotatable storage member 132. In different embodiments, the automated dispensing mechanism may comprise a cap-actuation mechanism, a container-opening mechanism, a tilting mechanism, a shaking mechanism, a metering mechanism, an indexing mechanism, a discharge-guiding mechanism, or any combination thereof. Dispensed medication may be directed to a user-accessible receiving region, chute, tray, receptacle, or outlet region of the apparatus 100. Following such automated dispensing, the controller may cause the movable cover 114 to return to the closed position and may perform the force-sensor-based verification and audit operations described herein. The automated dispensing mechanism is not separately shown in FIG. 16.

At optional step 212, the selected medication container 136 is replaced into the rotatable storage member 132. In the illustrated embodiment, step 212 is performed manually by the user after medication has been removed from the medication container 136. The selected medication container 136 may be returned into the compartment of the rotatable storage member 132 from which it was removed. In some embodiments, step 212 occurs before the controller commands closure of the movable cover 114. Step 212 is optional because certain implementations may permit removal of the selected medication container 136 without immediate replacement.

At step 214, the controller causes the second actuator 130 to move the movable cover 114 from the open position back to the closed position. In the illustrated embodiment, the second actuator 130 again drives the drive member 140, which engages the plurality of drive features 114A to return the movable cover 114 to the closed position. Step 214 may occur automatically after a predetermined time interval, after the controller detects a closure condition, after a user input, or after another suitable trigger. Step 214 therefore re-establishes closure of the access region following user access to the selected medication container 136.

At step 216, the controller detects vertical load by the plurality of force sensors 150. In the illustrated embodiment, the plurality of force sensors 150 is disposed relative to the base 152 and produces sensor outputs indicative of a vertical load transmitted through the apparatus 100. The controller reads the outputs of the plurality of force sensors 150 and obtains one or more load measurements associated with the dispensing event. In some embodiments, the controller reads a baseline load measurement and a subsequent load measurement for comparison. Step 216 therefore provides sensed load data usable for dispensing verification.

At step 218, the controller determines a weight delta corresponding to removal of medication. In the illustrated embodiment, the controller processes the sensor outputs obtained from the plurality of force sensors 150 and determines a difference between load measurements acquired at different times during the dispensing routine. The determined weight delta may correspond to removal of the selected medication container 136, removal of medication from the selected medication container 136, or another change in weight associated with the dispensing event. In some embodiments, the controller performs baseline correction, filtering, or another signal-processing operation when determining the weight delta. Step 218 therefore provides a derived dispensing-verification value from the force-sensor outputs.

At decision step 220, the controller compares the weight delta with an expected weight delta. In the illustrated embodiment, the expected weight delta may correspond to the selected medication, the selected medication container 136, a scheduled dose, or another expected dispensing condition. The controller determines whether the weight delta obtained at step 218 sufficiently matches the expected weight delta. A “Yes” outcome indicates that the measured change is consistent with the expected dispensing event, and a “No” outcome indicates a discrepancy. Step 220 therefore provides a verification decision based on controller comparison of measured and expected dispensing-related weight values.

At optional step 222, the controller generates a verification failure alert. In the illustrated embodiment, step 222 is reached when the comparison performed at step 220 yields a “No” outcome. The controller may generate an alert, flag, notification, or error condition indicating that the dispensing event did not satisfy an expected verification condition. In some embodiments, the controller stores the alert locally. In other embodiments, the controller transmits the alert through a network interface to a remote system or a caregiver device. Step 222 is optional because some implementations may record the discrepancy without generating a separate alert output.

At step 224, the controller generates an audit record. In the illustrated embodiment, the controller writes the audit record to a non-transitory storage medium operatively coupled to the controller. The audit record may include one or more of a timestamp, an identifier of the medication, an identifier of the selected medication container 136, the determined weight delta, a verification result, an alert condition, or another event-related datum. In some embodiments, the audit record comprises a cryptographically signed audit record. In some embodiments, the controller also transmits the audit record, or information derived therefrom, through a network interface to a remote system. Step 224 therefore provides persistent recording of the dispensing event and its verification status.

At step 226, the dispensing routine ends. In the illustrated embodiment, step 226 corresponds to completion of the controlled dispensing event after closure of the movable cover 114, performance of force-sensor-based verification, generation of any optional verification failure alert, and generation of the audit record. The controller may thereafter return the apparatus 100 to an idle or standby state pending a subsequent dispensing routine.

In the illustrated embodiment, FIG. 16 is shown as a generally top-to-bottom method flow. Optional steps 202, 204, 212, and 222 are shown in dashed-line boxes to indicate that those steps may be included in some embodiments and omitted in other embodiments. Decision step 220 is shown as a diamond to indicate a conditional comparison operation that may direct the method flow either directly to audit-record generation at step 224 or first to optional generation of the verification failure alert at step 222. Although the steps are shown in a particular sequence in FIG. 16, one or more steps may be omitted, repeated, combined, subdivided, or performed in another order in other embodiments.

The foregoing description of the embodiments has been presented for purposes of illustration and description. It is not intended to be exhaustive or to limit the technology disclosed to the precise forms described. Modifications and variations are possible in light of the above teachings or may be acquired from practice of the technology disclosed.

The embodiments described herein are illustrative only. Features described in connection with one embodiment may be combined with features described in connection with another embodiment unless such combination is expressly excluded or would be technically incompatible. Likewise, components illustrated as separate parts in one embodiment may be integrated into a single component in another embodiment, and components illustrated as a single component in one embodiment may be implemented as multiple components in another embodiment.

Mechanical, electromechanical, and structural elements described herein may be varied in form while remaining within the scope of the technology disclosed. By way of example, housings, support structures, movable covers, rotatable storage members, couplers, partitions, drive members, bearings, mounting brackets, force sensors, and actuator arrangements may differ in geometry, material, fastening arrangement, mounting position, or relative orientation from those expressly illustrated and described.

To the extent that control operations are described herein, such operations may be carried out by one or more controllers, processors, control circuits, or other electronic control components, and may be implemented using hardware, firmware, software, or any suitable combination thereof. Storage of schedules, audit information, verification results, alert conditions, or other operational data may be performed using any suitable non-transitory storage medium.

Any methods described herein, including methods described with reference to flow diagrams, should not be construed as requiring that the steps be performed in the particular order shown or described unless such order is expressly required by the claims or by the context. Steps may be omitted, repeated, combined, subdivided, or performed in a different order in other embodiments.

Directional and positional terms such as upper, lower, top, bottom, front, rear, side, inward, outward, above, below, vertical, and horizontal are used for convenience with reference to the illustrated embodiments and are not intended to require a particular orientation unless expressly stated otherwise.

Use of singular terms such as “a,” “an,” and “the” includes plural referents unless the context clearly indicates otherwise. Similarly, use of plural terms includes the singular unless the context clearly indicates otherwise.

No feature, component, step, or limitation described in the specification should be understood as essential to all embodiments unless expressly stated to be so. The scope of the technology disclosed is defined by the claims, and all changes that come within the meaning and range of equivalency of the claims are intended to be embraced therein.

In some implementations, one or more clauses described in this section, or elements thereof, can be implemented in the form of a medication storage and dispensing apparatus including structural, electromechanical, sensing, and control components configured to perform the recited operations. In some implementations, one or more clauses described in this section, or elements thereof, can be implemented using a controller operatively coupled to a non-transitory storage medium storing instructions executable by the controller to perform one or more of the method steps described herein. In further implementations, one or more clauses described in this section, or elements thereof, can be implemented using a combination of mechanical components, electromechanical actuators, sensing components, and controller-executed instructions configured to carry out the specific techniques described herein.

The clauses described in this section can be combined as features. In the interest of conciseness, combinations of features are not individually enumerated and are not repeated with each base set of features. The reader will understand how features identified in the clauses described in this section can be combined with other disclosed features, implementations, embodiments, systems, apparatuses, and methods described elsewhere in this application. These clauses are not intended to be mutually exclusive, exhaustive, or restrictive, and the technology disclosed is not limited to these clauses but rather encompasses combinations, modifications, and variations within the scope of the claimed technology and its equivalents.

In some implementations, one or more clauses described in this section can be embodied in a system including a controller and memory storing instructions executable by the controller to perform one or more of the operations described herein. In some implementations, one or more clauses described in this section can be embodied in a non-transitory storage medium storing instructions executable by a controller or processor of the apparatus to perform one or more of the operations described herein. In some implementations, one or more clauses described in this section can be embodied in an apparatus including one or more actuators, one or more sensing components, and one or more control components configured to perform one or more of the clauses described in this section.

### Clauses

1. A medication storage and dispensing apparatus comprising:
a housing;
a rotatable storage member disposed within the housing and rotatable about a generally vertical axis, the rotatable storage member configured to carry a plurality of medication containers at angularly distributed positions;
a movable cover disposed within the housing, the movable cover comprising a plurality of drive features disposed on an outer wall of the movable cover and being movable about the generally vertical axis between a closed position obscuring an access region of the apparatus and an open position exposing the access region;
a first actuator configured to rotate the rotatable storage member about the generally vertical axis to align a selected medication container of the plurality of medication containers with the access region; and
a second actuator distinct from the first actuator, the second actuator being operatively coupled to a drive member having a plurality of engagement features complementary to and engaged with the plurality of drive features of the movable cover to define a meshing engagement, such that rotational motion imparted by the second actuator to the drive member is transmitted through the meshing engagement to the movable cover to drive the movable cover about the generally vertical axis between the closed position and the open position.

2. The apparatus of clause 1, further comprising one or more removable partitions removably engageable with the rotatable storage member to subdivide the rotatable storage member into one or more compartments, each compartment configured to receive a medication container of the plurality of medication containers.

3. The apparatus of clause 2, wherein the rotatable storage member is reconfigurable between a first configuration having a first number of compartments and a second configuration having a second number of compartments different from the first number, the reconfiguration being effected by addition or removal of the one or more removable partitions without removal of the rotatable storage member from the housing.

4. The apparatus of clause 1, further comprising:
a female coupler disposed on an underside region of the rotatable storage member; and
a corresponding male coupler carried by an output shaft of the first actuator; wherein the corresponding male coupler comprises a generally frusto-conical body having a plurality of circumferential engagement features disposed on an outer surface of the generally frusto-conical body, wherein the female coupler comprises a generally dome-shaped body defining an operative inner cavity configured to receive the corresponding male coupler therein, and wherein the plurality of circumferential engagement features cooperates with the operative inner cavity to define a keyed engagement that re-establishes a predetermined angular relationship between the rotatable storage member and the first actuator upon each disassembly and reassembly of the rotatable storage member from the housing without requiring a user-administered calibration procedure.

5. The apparatus of clause 1, further comprising a plurality of force sensors disposed beneath at least a portion of the housing at angularly distributed positions about the generally vertical axis, the plurality of force sensors being configured to detect a vertical load transmitted from one or more components of the apparatus supported above the plurality of force sensors.

6. The apparatus of clause 1, wherein the housing comprises a top cover assembly, an upper housing, and a lower housing, the top cover assembly defining a cutout configured to expose a portion of the movable cover.

7. The apparatus of clause 1, further comprising a child-resistant cap removably mountable on each medication container of the plurality of medication containers, the child-resistant cap configured to require a deliberate combined manipulation by a user for disengagement.

8. A method of dispensing medication using a medication storage and dispensing apparatus, the apparatus comprising a housing defining an access region, a rotatable storage member disposed within the housing and rotatable about a generally vertical axis, a movable cover disposed within the housing and comprising a plurality of drive features disposed on an outer wall of the movable cover, a first actuator, a second actuator distinct from the first actuator, and a drive member operatively coupled to the second actuator and having a plurality of engagement features complementary to and engaged with the plurality of drive features of the movable cover to define a meshing engagement, the method comprising:
rotating, by the first actuator, the rotatable storage member about the generally vertical axis to align a selected medication container of a plurality of medication containers carried by the rotatable storage member with the access region; and
moving, by the second actuator, the movable cover from a closed position obscuring the access region to an open position exposing the access region by transmitting rotational motion from the drive member through the meshing engagement to the movable cover, thereby driving the movable cover about the generally vertical axis between the closed position and the open position to expose the selected medication container through the access region.

9. The method of clause 8, further comprising applying, by the second actuator in a de-energized state, a retention torque to the movable cover in the closed position.

10. The method of clause 8, further comprising, after dispensing of the medication, moving by the second actuator the movable cover from the open position back to the closed position.

11. The method of clause 8, further comprising sensing, by a plurality of force sensors, a change in vertical load corresponding to removal of a medication container from the apparatus.

12. The method of clause 11, further comprising generating an audit record of a dispensing event corresponding to the change in vertical load.

13. The method of clause 8, wherein moving the movable cover comprises engaging, by a drive member operatively coupled to the second actuator, a plurality of drive features disposed on an outer wall of the movable cover.

14. The method of clause 8, further comprising establishing a home angular reference for the rotatable storage member by cooperation of an angular reference feature on a periphery of the rotatable storage member with a fixed reference structure fixedly mounted relative to the housing of the apparatus.

15. The method of clause 8, further comprising receiving the selected medication container from the rotatable storage member through the access region in the open position of the movable cover and replacing the selected medication container into the rotatable storage member through the access region prior to the moving of the movable cover from the open position back to the closed position.

16. A medication storage and dispensing apparatus comprising:
a housing including an upper housing and a lower housing;
a rotatable storage member disposed within the housing and rotatable about a generally vertical axis, the rotatable storage member configured to carry a plurality of medication containers at angularly distributed positions;
a movable cover disposed within the housing, the movable cover comprising a plurality of drive features disposed on an outer wall of the movable cover and being movable about the generally vertical axis between a closed position obscuring an access region of the apparatus and an open position exposing the access region; a first actuator configured to rotate the rotatable storage member;
a second actuator distinct from the first actuator, the second actuator being operatively coupled to a drive member having a plurality of engagement features complementary to and engaged with the plurality of drive features of the movable cover to define a meshing engagement, such that motion imparted by the second actuator to the drive member is transmitted through the meshing engagement to drive the movable cover between the closed position and the open position;
a support structure disposed within the housing and configured to receive the rotatable storage member;
a female coupler disposed on an underside region of the rotatable storage member and defining an operative inner cavity;
a corresponding male coupler carried by an output shaft of the first actuator and comprising a generally frusto-conical body received within the operative inner cavity of the female coupler through a keyed engagement, the keyed engagement re-establishing a predetermined angular relationship between the rotatable storage member and the first actuator upon each disassembly and reassembly of the rotatable storage member from the housing without requiring a user-administered calibration procedure;
an angular reference feature disposed on a periphery of the rotatable storage member; a fixed reference structure fixedly mounted relative to the housing and arranged to cooperate with the angular reference feature to define a home angular reference for the rotatable storage member;
the lower housing carrying a front face having an opening receiving a user-interface display and a side face having a plurality of perforations;
a base forming a lower boundary of the apparatus, the base comprising a battery-receiving region defined by one or more upstanding walls extending upwardly from an upper surface of the base;
a battery received within the battery-receiving region;
a plurality of force sensors disposed on the upper surface of the base at angularly distributed positions about the generally vertical axis and configured to detect a vertical load transmitted from the apparatus through the plurality of force sensors to the base; and an actuator mounting bracket disposed within the lower housing and receiving the first actuator therein.

17. The apparatus of clause 16, further comprising:
a central bushing disposed on the support structure and defining a central through-passage arranged generally coaxially with the generally vertical axis;
a central projecting element extending downwardly from a base plate of the rotatable storage member and received within the central through-passage of the central bushing; and
a rotational bearing disposed within the central bushing and rotatably supporting the central projecting element for rotation about the generally vertical axis; wherein the female coupler is coupled to a lower end of the central projecting element by way of a fastener disposed at the lower end such that rotation of the corresponding male coupler is transmitted through the keyed engagement into the female coupler, through the fastener into the central projecting element, and through the central projecting element into the base plate of the rotatable storage member.

18. The apparatus of clause 16, further comprising a depending support element extending downwardly from an inner surface of an upper wall portion of the upper housing, the depending support element configured to rotatably support an upper end of the rotatable storage member through a second rotational bearing interposed between an operative top end of a central pillar of the rotatable storage member and the depending support element.

19. The apparatus of clause 16, further comprising an actuator mounting bracket disposed within the lower housing, the actuator mounting bracket including a generally planar base plate and a pair of upstanding bracket portions extending upwardly from the generally planar base plate and cooperatively defining a receiving region therebetween configured to receive and retain the first actuator in a predetermined orientation.

20. The apparatus of clause 16, further comprising a lighting floor positioned between the support structure and the lower housing, the lighting floor defining a central aperture aligned with the generally vertical axis and configured to receive the output shaft of the first actuator therethrough.

21. The apparatus of clause 16, wherein the support structure comprises an actuator-receiving recess defined at a peripheral region thereof and configured to receive the second actuator and a drive member of the second actuator therewithin.

22. The apparatus of clause 16, further comprising a top cover assembly mounted above an upper housing of the housing, the top cover assembly including:
a generally planar body defining a cutout configured to expose a portion of the movable cover;
a depending wall extending downwardly from an underside of the generally planar body along an inner boundary of the cutout; and
a plurality of depending projections extending downwardly from the underside of the generally planar body and configured to engage the upper housing to retain the top cover assembly on the upper housing.

23. A method of dispensing medication using a medication storage and dispensing apparatus, the apparatus comprising a housing, a rotatable storage member, a movable cover, a first actuator, a second actuator, a support structure, a central bushing, a central projecting element extending downwardly from the rotatable storage member, a female coupler, and a corresponding male coupler, the method comprising:
rotating, by the first actuator, the rotatable storage member about a generally vertical axis by transmitting rotation from the first actuator through the corresponding male coupler into the female coupler and through the central projecting element into the rotatable storage member to align a selected medication container with an access region of the apparatus; and
moving, by the second actuator, the movable cover from a closed position to an open position to expose the selected medication container through the access region.

24. The method of clause 23, further comprising:
removing the rotatable storage member from the housing by disengaging the female coupler from the corresponding male coupler; and
reassembling the rotatable storage member into the housing by re-engaging the female coupler with the corresponding male coupler through the keyed engagement, the re-engagement re-establishing a predetermined angular relationship between the rotatable storage member and the first actuator without a user-administered calibration procedure.

25. The method of clause 23, further comprising verifying an angular position of the rotatable storage member by cooperation of an angular reference feature on a periphery of the rotatable storage member with a fixed reference structure fixedly mounted relative to the housing.

26. The method of clause 23, further comprising performing a homing routine upon power-up of the apparatus, the homing routine comprising rotating the rotatable storage member through a predetermined angular range and detecting alignment of an angular reference feature on the rotatable storage member with a fixed reference structure fixedly mounted relative to the housing.

27. The method of clause 23, further comprising:
measuring, by a plurality of force sensors, a vertical load before and after a dispensing event corresponding to removal of the selected medication container from the apparatus; and
determining a weight delta from the measured vertical loads to confirm removal of the selected medication container.

28. The method of clause 27, further comprising generating a verification failure alert when the determined weight delta does not match an expected weight delta corresponding to the selected medication container.

28. A medication storage and dispensing apparatus comprising:
a housing including an upper housing and a lower housing;
a rotatable storage member disposed within the housing and rotatable about a generally vertical axis, the rotatable storage member configured to carry a plurality of medication containers at angularly distributed positions;
a movable cover disposed within the housing, the movable cover comprising a plurality of drive features disposed on an outer wall of the movable cover and being movable about the generally vertical axis between a closed position obscuring an access region of the apparatus and an open position exposing the access region;
a first actuator configured to rotate the rotatable storage member;
a second actuator distinct from the first actuator and configured to move the movable cover;
a support structure disposed within the housing and configured to receive the rotatable storage member;
a female coupler disposed on an underside region of the rotatable storage member; a corresponding male coupler carried by an output shaft of the first actuator and engageable with the female coupler;
the lower housing carrying a front face with an opening receiving a user-interface display and a side face with a plurality of perforations;
a base forming a lower boundary of the apparatus, the base comprising a battery-receiving region defined by one or more upstanding walls extending upwardly from an upper surface of the base;
a battery received within the battery-receiving region;
a plurality of force sensors disposed on the upper surface of the base at angularly distributed positions about the generally vertical axis and configured to detect a vertical load transmitted from the apparatus through the plurality of force sensors to the base; and
an actuator mounting bracket disposed within the lower housing and receiving the first actuator therein.

29. The apparatus of clause 28, wherein the movable cover comprises a recessed contour disposed at an upper edge region thereof and configured to engage a depending support element of the upper housing, the depending support element rotatably supporting the movable cover at the recessed contour during movement of the movable cover between the closed position and the open position.

30. The apparatus of clause 28, wherein the corresponding male coupler comprises a generally frusto-conical body and includes a plurality of circumferential engagement features disposed on an outer surface of the generally frusto-conical body, and wherein the female coupler comprises a generally dome-shaped body defining an operative inner cavity configured to receive the corresponding male coupler therein in a keyed engagement.

31. The apparatus of clause 28, wherein the rotatable storage member comprises:
a base plate having a plurality of anchor positions disposed circumferentially thereon; and
a central pillar extending upwardly from the base plate, the central pillar including a plurality of vertical cutouts extending circumferentially about the central pillar;
wherein the plurality of anchor positions and the plurality of vertical cutouts are arranged to cooperatively receive one or more removable partitions, each removable partition including a plurality of engagement legs configured to engage a corresponding anchor position and a pillar-engagement edge configured to engage a corresponding vertical cutout.

32. A method of dispensing medication using a medication storage and dispensing apparatus, the method comprising:
rotating, by a first actuator of the apparatus, a rotatable storage member of the apparatus about a generally vertical axis to align a selected medication container with an access region of the apparatus;
moving, by a second actuator of the apparatus, a movable cover of the apparatus from a closed position obscuring the access region to an open position exposing the access region;
receiving the selected medication container from the rotatable storage member through the access region in the open position;
detecting, by a plurality of force sensors of the apparatus, a weight delta corresponding to removal of the selected medication container; and
moving, by the second actuator, the movable cover from the open position back to the closed position after replacement of the selected medication container into the rotatable storage member.

33. The method of clause 32, further comprising:
comparing the detected weight delta with an expected weight delta corresponding to the selected medication container; and
generating an audit record of the dispensing event and a verification result based on the comparison.

34. The method of clause 32, further comprising reconfiguring the rotatable storage member between a first configuration having a first number of compartments and a second configuration having a second number of compartments different from the first number, the reconfiguration being effected by addition or removal of one or more removable partitions without removal of the rotatable storage member from the medication storage and dispensing apparatus.

35. The method of clause 32, further comprising retaining, by the second actuator in a de-energized state, the movable cover in the closed position with a retention torque sufficient to resist user attempts to manually displace the movable cover, thereby providing a child-resistant retention mechanism that operates independently of an angular position of the rotatable storage member.

The above-described hardware description is a non-limiting example of corresponding structure for performing the functionality described herein.

Numerous modifications and variations of the present disclosure are possible in light of the above teachings. It is therefore to be understood that the technology disclosed may be practiced otherwise than as specifically described herein.

What is claimed is:

1. A medication storage and dispensing apparatus comprising:
a housing;
a rotatable storage member disposed within the housing and rotatable about a generally vertical axis, the rotatable storage member configured to carry a plurality of medication containers at angularly distributed positions;
a movable cover disposed within the housing, the movable cover comprising a plurality of drive features disposed on an outer wall of the movable cover and being movable about the generally vertical axis between a closed position obscuring an access region of the apparatus and an open position exposing the access region;
a first actuator configured to rotate the rotatable storage member about the generally vertical axis to align a selected medication container of the plurality of medication containers with the access region; and
a second actuator distinct from the first actuator, the second actuator being operatively coupled to a drive member having a plurality of engagement features complementary to and engaged with the plurality of drive features of the movable cover to define a meshing engagement, such that rotational motion imparted by the second actuator to the drive member is transmitted through the meshing engagement to the movable cover to drive the movable cover about the generally vertical axis between the closed position and the open position.

2. The apparatus of claim 1, further comprising one or more removable partitions removably engageable with the rotatable storage member to subdivide the rotatable storage member into one or more compartments, each compartment configured to receive a medication container of the plurality of medication containers.

3. The apparatus of claim 2, wherein the rotatable storage member is reconfigurable between a first configuration having a first number of compartments and a second configuration having a second number of compartments different from the first number by addition or removal of the one or more removable partitions without removal of the rotatable storage member from the housing.

4. The apparatus of claim 1, further comprising:
a female coupler disposed on an underside region of the rotatable storage member; and
a corresponding male coupler carried by an output shaft of the first actuator;
wherein the corresponding male coupler comprises a generally frusto-conical body having a plurality of circumferential engagement features disposed on an outer surface of the generally frusto-conical body, wherein the female coupler comprises a generally dome-shaped body defining an operative inner cavity configured to receive the corresponding male coupler therein, and wherein the plurality of circumferential engagement features cooperates with the operative inner cavity to define a keyed engagement that re-establishes a predetermined angular relationship between the rotatable storage member and the first actuator upon each disassembly and reassembly of the rotatable storage member from the housing without requiring a user-administered calibration procedure.

5. The apparatus of claim 1, further comprising a plurality of force sensors disposed beneath at least a portion of the housing at angularly distributed positions about the generally vertical axis, the plurality of force sensors being configured to detect a vertical load transmitted from one or more components of the apparatus supported above the plurality of force sensors.

6. The apparatus of claim 1, wherein the housing comprises a top cover assembly, an upper housing, and a lower housing, the top cover assembly defining a cutout configured to expose a portion of the movable cover.

7. The apparatus of claim 1, further comprising a child-resistant cap removably mountable on each medication container of the plurality of medication containers, the child-resistant cap configured to require a deliberate combined manipulation by a user for disengagement.

8. The apparatus of claim 1, further comprising a support structure disposed within the housing, the support structure configured to receive the rotatable storage member, the support structure comprising an actuator-receiving recess defined at a peripheral region of the support structure, the actuator-receiving recess being configured to receive the second actuator and the drive member therewithin.

9. The apparatus of claim 8, wherein the housing comprises an upper housing, and wherein the movable cover further comprises a recessed contour disposed at an upper edge region of the movable cover, the plurality of drive features being disposed along a lower circumferential portion of the outer wall of the movable cover, the recessed contour being configured to engage a depending support element of the upper housing such that the depending support element rotatably supports the movable cover at the recessed contour during powered rotation of the movable cover about the generally vertical axis by the second actuator between the closed position and the open position.

10. The apparatus of claim 1, further comprising:
an angular reference feature disposed on a periphery of the rotatable storage member; and
a fixed reference structure fixedly mounted relative to the housing and arranged to cooperate with the angular reference feature to define a home angular reference for the rotatable storage member;
wherein the home angular reference is re-establishable upon each disassembly and reassembly of the rotatable storage member from the housing without requiring a user-administered calibration procedure.

11. A method of dispensing medication using a medication storage and dispensing apparatus, the apparatus comprising a housing defining an access region, a rotatable storage member disposed within the housing and rotatable about a generally vertical axis, a movable cover disposed within the housing and comprising a plurality of drive features disposed on an outer wall of the movable cover, a first actuator, a second actuator distinct from the first actuator, and a drive member operatively coupled to the second actuator and having a plurality of engagement features complementary to and engaged with the plurality of drive features of the movable cover to define a meshing engagement, the method comprising:
rotating, by the first actuator, the rotatable storage member about the generally vertical axis to align a selected medication container of a plurality of medication containers carried by the rotatable storage member with the access region; and
moving, by the second actuator, the movable cover from a closed position obscuring the access region to an open position exposing the access region by transmitting rotational motion from the drive member through the meshing engagement to the movable cover, thereby driving the movable cover about the generally vertical axis between the closed position and the open position to expose the selected medication container through the access region.

12. The method of claim 11, further comprising maintaining, by the second actuator in a de-energized state, a retention torque applied to the movable cover while the movable cover is in the closed position.

13. The method of claim 11, further comprising, after dispensing of medication, moving the movable cover from the open position back to the closed position by the second actuator.

14. The method of claim 11, further comprising sensing, by a plurality of force sensors disposed beneath at least a portion of the housing at angularly distributed positions about the generally vertical axis, a change in vertical load corresponding to removal of the selected medication container from the apparatus.

15. The method of claim 14, further comprising comparing the change in vertical load with an expected weight delta corresponding to the selected medication container and generating an audit record of a dispensing event based on the comparison, wherein the plurality of force sensors detects a baseline vertical load after establishment of a home angular reference for the rotatable storage member by cooperation of an angular reference feature on a periphery of the rotatable storage member with a fixed reference structure fixedly mounted relative to the housing, and wherein the change in vertical load is determined relative to the baseline vertical load.

16. The method of claim 11, further comprising performing a homing routine for the rotatable storage member, the homing routine comprising rotating the rotatable storage member by the first actuator through a predetermined angular range and detecting alignment of an angular reference feature disposed on a periphery of the rotatable storage member with a fixed reference structure fixedly mounted relative to the housing.

17. A medication storage and dispensing apparatus comprising:
a housing including an upper housing and a lower housing;
a rotatable storage member disposed within the housing and rotatable about a generally vertical axis, the rotatable storage member configured to carry a plurality of medication containers at angularly distributed positions;
a movable cover disposed within the housing, the movable cover comprising a plurality of drive features disposed on an outer wall of the movable cover and being movable about the generally vertical axis between a closed position obscuring an access region of the apparatus and an open position exposing the access region;
a first actuator configured to rotate the rotatable storage member;
a second actuator distinct from the first actuator, the second actuator being operatively coupled to a drive member having a plurality of engagement features complementary to and engaged with the plurality of drive features of the movable cover to define a meshing engagement, such that motion imparted by the second actuator to the drive member is transmitted through the meshing engagement to drive the movable cover between the closed position and the open position;
a support structure disposed within the housing and configured to receive the rotatable storage member;
a female coupler disposed on an underside region of the rotatable storage member and defining an operative inner cavity;
a corresponding male coupler carried by an output shaft of the first actuator and comprising a generally frusto-conical body received within the operative inner cavity of the female coupler through a keyed engagement, the keyed engagement re-establishing a predetermined angular relationship between the rotatable storage member and the first actuator upon each disassembly and reassembly of the rotatable storage member from the housing without requiring a user-administered calibration procedure;
an angular reference feature disposed on a periphery of the rotatable storage member;
a fixed reference structure fixedly mounted relative to the housing and arranged to cooperate with the angular reference feature to define a home angular reference for the rotatable storage member;
the lower housing carrying a front face having an opening receiving a user-interface display and a side face having a plurality of perforations;
a base forming a lower boundary of the apparatus, the base comprising a battery-receiving region defined by one or more upstanding walls extending upwardly from an upper surface of the base;
a battery received within the battery-receiving region;
a plurality of force sensors disposed on the upper surface of the base at angularly distributed positions about the generally vertical axis and configured to detect a vertical load transmitted from the apparatus through the plurality of force sensors to the base; and
an actuator mounting bracket disposed within the lower housing and receiving the first actuator therein.

18. The apparatus of claim 17, wherein the movable cover further comprises a recessed contour disposed at an upper edge region of the movable cover and configured to engage a depending support element of the upper housing, the depending support element extending downwardly from an inner surface of an upper wall portion of the upper housing and rotatably supporting the movable cover at the recessed contour during powered rotation of the movable cover about the generally vertical axis by the second actuator between the closed position and the open position.

19. The apparatus of claim 17, further comprising:
a central bushing disposed on the support structure and defining a central through-passage arranged generally coaxially with the generally vertical axis;
a central projecting element extending downwardly from a base plate of the rotatable storage member and received within the central through-passage of the central bushing; and
a rotational bearing disposed within the central bushing and rotatably supporting the central projecting element for rotation about the generally vertical axis;
wherein the female coupler is coupled to a lower end of the central projecting element by way of a fastener disposed at the lower end such that rotation of the corresponding male coupler is transmitted through the keyed engagement into the female coupler, through the fastener into the central projecting element, and through the central projecting element into the base plate of the rotatable storage member.

20. The apparatus of claim 17, wherein the rotatable storage member comprises:
a base plate having a plurality of anchor positions disposed circumferentially thereon; and
a central pillar extending upwardly from the base plate, the central pillar including a plurality of vertical cutouts extending circumferentially about the central pillar;
wherein the plurality of anchor positions and the plurality of vertical cutouts are arranged to cooperatively receive one or more removable partitions, each removable partition including a plurality of engagement legs engageable with a corresponding anchor position and a pillar-engagement edge engageable with a corresponding vertical cutout.
